SEQUENCE LISTING (34 sequences)

SEQ ID NO: 1 - DNA, 21 bp, synthetic construct
  source 1..21: mol_type = other DNA
ttttttttttttttttttttt

SEQ ID NO: 2 - DNA, 21 bp, synthetic construct
  source 1..21: mol_type = other DNA
ttttttttttttttttttttt

SEQ ID NO: 3 - DNA, 22 bp, synthetic construct
  source 1..22: mol_type = other DNA
tttttttttttttttttttttt

SEQ ID NO: 4 - DNA, 23 bp, synthetic construct
  source 1..23: mol_type = other DNA
ttttttttttttttttttttttt

SEQ ID NO: 5 - DNA, 25 bp, synthetic construct
  source 1..25: mol_type = other DNA
ttttttttttttttttttttttttt

SEQ ID NO: 6 - DNA, 22 bp, synthetic construct
  source 1..22: mol_type = other DNA
tttttttttttttttttttttt

SEQ ID NO: 7 - DNA, 23 bp, synthetic construct
  source 1..23: mol_type = other DNA
ttttttttttttttttttttttt

SEQ ID NO: 8 - DNA, 21 bp, synthetic construct
  source 1..21: mol_type = other DNA
ttttttttttttttttttttt

SEQ ID NO: 9 - DNA, 21 bp, synthetic construct
  source 1..21: mol_type = other DNA
cactgttgttttcgtttggtg

SEQ ID NO: 10 - DNA, 20 bp, synthetic construct
  source 1..20: mol_type = other DNA
gtcagccttcacccactcat

SEQ ID NO: 11 - DNA, 20 bp, synthetic construct
  source 1..20: mol_type = other DNA
ttccttcagtgcacttagcc

SEQ ID NO: 12 - DNA, 20 bp, synthetic construct
  source 1..20: mol_type = other DNA
atgccaggagctgaaactgt

SEQ ID NO: 13 - DNA, 20 bp, synthetic construct
  source 1..20: mol_type = other DNA
aggctttcaggattgtggtt

SEQ ID NO: 14 - DNA, 21 bp, synthetic construct
  source 1..21: mol_type = other DNA
ctccccaataacacattgtcc

SEQ ID NO: 15 - DNA, 20 bp, synthetic construct
  source 1..20: mol_type = other DNA
aaggaatggtgggcaaagta

SEQ ID NO: 16 - DNA, 21 bp, synthetic construct
  source 1..21: mol_type = other DNA
ccttcgggaaactcttctttc

SEQ ID NO: 17 - DNA, 20 bp, synthetic construct
  source 1..20: mol_type = other DNA
tacaccgtgagctgtgctct

SEQ ID NO: 18 - DNA, 19 bp, synthetic construct
  source 1..19: mol_type = other DNA
aggcccaggcctttagttt

SEQ ID NO: 19 - DNA, 20 bp, synthetic construct
  source 1..20: mol_type = other DNA
ttgtcctggatgatgttgct

SEQ ID NO: 20 - DNA, 20 bp, synthetic construct
  source 1..20: mol_type = other DNA
acggttgctgaaaaccactg

SEQ ID NO: 21 - DNA, 20 bp, synthetic construct
  source 1..20: mol_type = other DNA
agctctggcttttggctttt

SEQ ID NO: 22 - DNA, 20 bp, synthetic construct
  source 1..20: mol_type = other DNA
tgcaaagcagccatactctg

SEQ ID NO: 23 - DNA, 20 bp, synthetic construct
  source 1..20: mol_type = other DNA
gtattccgcctttcttccaa

SEQ ID NO: 24 - DNA, 20 bp, synthetic construct
  source 1..20: mol_type = other DNA
ctcttccattttccccaatg

SEQ ID NO: 25 - DNA, 421 bp, synthetic construct
  source 1..421: mol_type = other DNA
taagaaatcctcttgcctttccctagtgagtatacaaaatacctactacagttactggcacatgttaccagcaaaccagcccctgtgcccttcatattcttgccaacactaagaacaaactatttaatctgtctcactgttgttttcgtttggtgaatacatttcaaaaaggttataatgtataaagtttgtacaaactctttttttttttttttttttttaaggtatgatgagtgggtgaaggctgacaggataatctggcctttggacaaaggtggaccaaagaaaaaacagaagaaaaaagctaaagtatgtttgtagatttatgtcctttataatatgtataggaattatccaaattgtattgtgtgttttttaagactttaagaacagttttaggtttataccaagattgagagca

SEQ ID NO: 26 - DNA, 421 bp, synthetic construct
  source 1..421: mol_type = other DNA
catggcccagtattttagaaaatgataatagcctagtagaaaagtcattatattctgtaatctgaatgtgttcttcttgccactatttggtgaatagacctatgtcacacatatgcagaaatacgtagttgctgataaatggtcaaagttatagaaggctattttccttcagtgcacttagccagctaatttgctgcttctttttttttttttttttttttaaacagtttcagctcctggcatcagctctattcaaatctggttcagattttacagctctgggtaagtattgcagtataatttatccagctagatgccctagcatttggaaggttggattttggtttcttttacgtgaaacatcaggtgagatgtttcagtcaaatagatgagcaaatgaagtttcaaggtatcatgcaaa

SEQ ID NO: 27 - DNA, 422 bp, synthetic construct
  source 1..422: mol_type = other DNA
cccacctcggcctcccaaagtgctgggattacaggtgtgagccaccgcgcctggccaatattttctttaatcatcaaatatttacagatctgtgaccacttaccttaaaacttatgaatggtctctttgtaccgtaataggctttcaggattgtggttaggtcatcgattttactgtctttcattataattatttgtgtattttttttttttttttttttttaggttcagatgattttatgccactagtaagaatccaaggacaatgtgttattggggagaagtagaaaaaggaaaatctggggtagcacctggcatgctctttctccaattttctactacttactcctattccccaaattctcccatcaaggaggaaatgaactctgagacagaagatgagttttcctcaaagcttgacca

SEQ ID NO: 28 - DNA, 423 bp, synthetic construct
  source 1..423: mol_type = other DNA
aattgttctagatgttaacaattccaaggatgctgaacagcagcccttattttcagtatgtgacattatctaacctataggagtggaagaacacaacaattttttttaacccaaggaatggtgggcaaagtaagattttgtagattaagatgtgatccactagtagtcattataattagcatatcagtttgtaattggtctttttttttttttttttttttttaggagatgaaagaagagtttcccgaaggtatgaagattgtcaagcatcaggtaagagagcattgtatatgagatgtgagttaaagttctggctttattttattttactgcttggtaagtaacaagtttgtgtctacagacacatttgttggactcttacttgcttagtcatacctaatctagttagttaggagtttcttc

SEQ ID NO: 29 - DNA, 425 bp, synthetic construct
  source 1..425: mol_type = other DNA
cgtctcccaaagtttattgaacatcttctcaaatcctgtttcaagcacaaaacttttaaccacattcagagggttttaaacgagctttgagatgggtcactagcttctcagaaaatccacatctaaacttctccagtacaccgtgagctgtgctctagccaaagaaaaatggaaagacagagaaaggagttctgagagcctttttttttttttttttttttttttaaggaaaaagaagaatttattcactcaagactaaaaactaaaggcctgggcctgagaactgaatcaggttagtgtgtttgtttgattgttttttttgctttctttgcgtttgagctagtccatgaaaagggaaaggaagggcggtgagagaggtggggaagttcagggcggtccttggcctgctttggggcagtgctcgg

SEQ ID NO: 30 - DNA, 422 bp, synthetic construct
  source 1..422: mol_type = other DNA
cttgatgaattgtctaatactactttggtccgatacaaaataaaaagtttaaattttctcatagcttttctgaaatgtcagtcatcttatgtttgtagtgattcttttaatgtttacttttgcgtttttttaaaaaagacttaggaaaagattgtcctggatgatgttgctattgggagataatgcaaattgctctaagattttttttttttttttttttttaaggtgacaaaaacttagttggttcagaagtaaaaatttatagcttggacccatctactcagtggttttcagcaaccgttataaatggaaacccagcatcaaaaactcttcaagtcaactgtgaggaggtaaagacaacaataactctttgtaagataactcgacaaagcatgataactatacaaagcatttatgatttt

SEQ ID NO: 31 - DNA, 423 bp, synthetic construct
  source 1..423: mol_type = other DNA
tgagttttatcctttgggcagttagcctatgctttacctccctaaccttttatctggctggatttaaaaattgatggaaatgtttaatgaaaaagaaatacaaagatatgtagttttattatttttttttctgagttgaatatccggataagaatatgtaacttacagtaagaagctctggcttttggcttttggtgtgatttttttttttttttttttttttacctttatgcgaacttcctttgcctttggtggggccacttctacctcttcaatagaaaatggtgcaccaggcttccagagtatggctgctttgcatctgatgacctgggaaatagaatacaggggcagaaagagaagctccttaagcttgggagttggaagattgctatattagtgattatagaatcattaaattggatt

SEQ ID NO: 32 - DNA, 421 bp, synthetic construct
  source 1..421: mol_type = other DNA
agcagcaattcgaaacaggattggagtctcgaaccagaaactgcagtttaccggggacaatagccccgcgggagtggggcggggccaaggagatgtgggcggggagaggacgggggtcagaagaccgagggcgacctcgaggcgagggcgggacagcggcggggtgtgaagtacgcatgcgtattccgcctttcttccaatttttttttttttttttttttacctacttttgtaagacatgcgccattggggaaaatggaagagaagttgcgagtctgtgagaccctgatcttctggtataactactcggcttttccaacttccttgtctcagtgtagatcgcgcggtaacccaggccactcagaagtcccgagacccgatgctttcgccagcgtctcggtctccaaggaaacgcgacgcc

SEQ ID NO: 33 - DNA, 10 bp, synthetic construct
  source 1..10: mol_type = other DNA
tatatatata

SEQ ID NO: 34 - DNA, 15 bp, synthetic construct
  source 1..15: mol_type = other DNA
gtcgtcgtcgtcgtc

(19) United States
(12) Patent Application Publication
(10) Pub. No.: US 2026/0139318 A1
(43) Pub. Date: May 21, 2026

(54) METHOD FOR DETERMINING MICROSATELLITE INSTABILITY STATUS, KITS AND USES THEREOF

(71) Applicants: UNIVERSITE DE POITIERS, POITIERS (FR); CENTRE HOSPITALIER UNIVERSITAIRE DE POITIERS, POITIERS (FR)

(72) Inventors: ULRICH CORTES, ROCHES-PRÉMARIE-ANDILLÉ (FR); BIRAMA NDIAYE, POITIERS (FR); LUCIE KARAYAN-TAPON, POITIERS (FR)

(21) Appl. No.: 19/129,790

(22) PCT Filed: Nov. 17, 2023
(86) PCT No.: PCT/EP2023/082157

§ 371 Date: May 14, 2025

(30) Foreign Application Priority Data
Nov. 17, 2022 (EP) 22306693.7

(51) Int. Cl. C12Q 1/6886; G16B 40/00

(52) U.S. Cl. CPC C12Q 1/6886; G16B 40/00; C12Q 2600/156; C12Q 2600/158

(57) ABSTRACT

The present invention relates to the field of genomics. The invention relates more particularly to new microsatellite markers and their uses, in particular for determining the microsatellite status of a tumor, in particular of a tumor from a human subject. The description provides methods for analyzing DNA microsatellite loci. It also relates to the tools, kits and systems that can be used to implement such an analysis.

### CROSS-REFERENCE TO RELATED APPLICATION

This application is the U.S. national stage application of International Patent Application No. PCT/EP2023/082157, filed Nov. 17, 2023.

### REFERENCE TO SEQUENCE LISTING

The Sequence Listing for this application is labeled “Seq-List.xml” which was created on Apr. 24, 2025, and is 33,807 bytes. The entire content of the sequence listing is incorporated herein by reference in its entirety.

### FIELD OF THE INVENTION

The present invention relates to the field of genomics. The invention relates more particularly to new microsatellite markers and their uses, in particular for determining the microsatellite status of a tumor, in particular of a tumor from a human subject. The description provides methods for analyzing DNA microsatellite loci. It also relates to the tools, kits and systems that can be used to implement such an analysis.

### BACKGROUND OF THE INVENTION

Microsatellites are short repeated DNA sequences, coding or non-coding, composed of one to six base motifs tandemly repeated, that are also called “short tandem repeats”. During replication, DNA slippage occurs at microsatellite loci, resulting in alteration in the number of repeats and in the total length of microsatellites. The MisMatch Repair system (MMR), encoded by MLH1, MSH2, PMS2 and MSH6 genes, is involved in repairing these errors [Evrard et al.].

Microsatellite Instability (MSI) is the molecular hallmark of a deficient MisMatch Repair system (dMMR). Indeed, the dMMR deficiency results in a mutator phenotype with progressive accumulation of genetic instability and is characterized, among other aspects, by widespread variations in the length of microsatellites. The MSI phenotype is most commonly caused by hypermethylation of the MLH1 gene promoter in tumors, leading to its loss of expression [Cunningham J M et al.]. Colorectal Cancers (CRCs) and Endometrial Cancers (ECs) are most associated neoplasms with sporadic dMMR (15 to 20% and 20 to 30% respectively) [Bonneville R et al., Levine DA]. A less common cause is the inherited transmission of a mutated allele to one of the MMR genes, leading to Hereditary Non-Polyposis Colorectal Cancer (HNPCC), also known as Lynch syndrome.

There are two different validated techniques for detecting the dMMR phenotype in daily practice: immunohistochemistry (IHC) and Microsatellite Instability-Polymerase Chain Reaction assay (“MSI-PCR”) (Luchini C et al.). The first method identifies the loss of expression of at least one of the four proteins (MLH1, MSH2, MSH6 or PMS2) of the MMR system which reflects its loss of function. The European Society of Medical Oncology (ESMO) guidelines recommend its use in first intention in any sporadic cancer type belonging to the Lynch syndrome spectrum. The other method reveals the consequences of dMMR phenotype, microsatellites instability, through the analysis of microsatellites loci. The MSI-PCR requires the amplification of loci comprising microsatellites of interest and analysis by capillary electrophoresis of the length of PCR-generated amplicons. The MSI phenotype is defined by the instability of at least two microsatellites out of five of a pentaplex panel compliant with Bethesda guidelines for HNPCC (Bacher et al., 2004): BAT-25, BAT-26, NR-21, NR-24, and NR-27 (also identified as “MONO-27”). The use of this panel has been recommended by international guidelines, in case of uncertainty about IHC, or in the case of loss of only one MMR protein [Luchini C et al.]. Furthermore, due to a significant percentage of false positive dMMR (10%) or MSI-PCR results enrolled in immunotherapy CRC trials, it is also recommended to systematically associate IHC and MSI-PCR before using any immunotherapeutic agent in metastatic dMMR CRC [Levine DA]. For other types of non-colorectal tumors, such as EC tumors, the sensitivity of these tests is lower as they exhibit smaller repeat number changes than CRC, leading to a less marked shift in microsatellite markers [Wang Y]. For this reason, it is essential to compare tumor tissue and non-tumor tissue during MSI-PCR testing in these tumors, and PCR is strongly recommended as a confirmation test in case of dMMR status by IHC or equivocal results [Evrard C et al.].

Several studies have recently emphasized that dMMR is predictive of response to immune checkpoint inhibitors (ICI) in many cancer types. Pembrolizumab, an anti-programmed cell death protein 1 (anti-PD-1) immunotherapeutic agent doubled the time of progression free survival (PFS) in patients in comparison with chemotherapy as a first line treatment for dMMR metastatic CRC in the phase III KEYNOTE-177 trial [Andre T et al.]. The on-going phase II GARNET trial has recently proved the efficacy of Dostarlimab, another anti-PD-1 immunotherapeutic agent in pre-treated dMMR/MSI metastatic ECs and other solid tumors. Considering these promising results, the US Food and Drug Administration (FDA) granted accelerated approval to Dostarlimab for dMMR EC following a platinum-containing regimen [Berton D et al.]. In addition, the phase II KEYNOTE-158 trial has proved the efficacy of Pembrolizumab as a second-line treatment for other types of dMMR cancers, such as EC, pancreatic and gastric cancers, and cholangiocarcinoma [Marabelle A et al.].

With the development of immunotherapy as a new therapeutic option, the determination of MMR status has clearly become a crucial point in cancer management. However, the conventional methods have diagnostic limitations, for example IHC cannot always detect loss of mutated proteins resulting from missense mutations and can have normal staining even for some protein-truncating mutations [Shia J. et al.]. Moreover, cost implications, time, and tissue consumption (especially when non-tumor tissue is needed) relating to IHC and MSI-PCR analyses may represent limiting factors; in turn these tests are rarely performed on tumors with low dMMR prevalence, such as prostate cancer which may also benefit from immunotherapy. The determination of MSI status by Next-Generation Sequencing (MSI-NGS) can be an interesting alternative since it permits simultaneous analysis of numerous samples and can be associated with the search of somatic variants of theragnostic interest such as KRAS, NRAS and BRAF mutations in CRCs or POLE and TP53 mutations in ECs. There are many published studies regarding MSI-NGS algorithms however these require the sequencing of large microsatellite panels that cannot be used in daily practice and consequently have not been approved by international guidelines yet [Long D R et al., Hempelmann J A et al., Middha S et al., and Trabucco S E et al.]. Therefore, there is a need for a highly sensitive and specific MSI-NGS method that can be used in daily practice.

Only a few studies have devised NGS methods for MSI determination but exclusively for colorectal cancer, as in Herbreteau G. et al. and Ratovomanana T et al. studies, nevertheless these methods may only be used for colorectal cancer and cannot be implemented with confidence in cancers with minimal microsatellite shift (such as e.g., endometrial cancer). In addition, most of these methods require comparison to a matched non-tumor sample. Hence, so far there has been no convincing progress for addressing MSI determination of cancer, in particular of non-colorectal cancers, by NGS.

To address this need for CRCs but also for other tumors such as ECs, inventors herein describe a novel set of biomarkers and a novel method, compatible with NGS, for determining microsatellite status, in particular MSI status, in a highly sensitive and specific manner.

### SUMMARY OF THE INVENTION

Detection of microsatellite instability (MSI) phenotype is becoming increasingly essential in recent years due to the development of immune checkpoint inhibitors as an effective therapy for MSI tumors. To date these treatments are available for colorectal cancers (CRCs) and endometrial cancers (ECs) but will soon expand to other tumor types, hence there is a need to ensure our capacity to detect MSI in a more timely and efficient manner.

New markers are presented herein that have a high sensitivity to determine the microsatellite stability (MSS) status or microsatellite instability (MSI) status of a tumor. These markers are more sensitive than the prevailing standard pentaplex panel (i.e., BAT-25, BAT-26, NR-21, NR-24 and MONO-27), while retaining specificity for MSI, in the context of colon cancer, but not only.

The present description relates in particular to a method of analyzing a set of microsatellite loci, for example a set of at least two or at least three, preferably a set of at least four microsatellite loci, of human DNA selected from the group comprising:
CABIO-P05, defined as a 21 T repeat located at 14q23.1 and starting at position chr14: 58359108,
CABIO-P07, defined as a 21 T repeat located at 7q32 and starting at position chr7: 131478596,
CABIO-E01, defined as a 22 T repeat located at Xq22.3 and starting at position chrX: 106849221,
CABIO-E03, defined as a 23 T repeat located at Xq21.2 and starting at position chrX: 85268269,
CABIO-E04, defined as a 25 T repeat located at 14q32.3 and starting at position chr14: 103574079,
CABIO-E05, defined as a 22 T repeat located at 2p11.2 and starting at position chr2: 86456417,
CABIO-E06, defined as a 23 T repeat located at 4q23 and starting at position chr4: 99216136, and
CABIO-E07, defined as a 21 T repeat located at 20p13 and starting at position chr20: 290564, with reference to the Homo sapiens reference genome assembly from Genome Reference Consortium human Build 38 patch release 14, also herein identified as GRCh38.p14 or simply as GRCh38.

The description further relates to a method of assessing the microsatellite stability (MSS) or microsatellite instability (MSI) status of a tumor comprising the steps of:
a) counting the number of indel(s) in at least two, for example at least three, preferably at least four microsatellite loci in a sample of tumor's DNA, wherein the tumor is preferably a human tumor and the microsatellite loci are preferably selected from the group comprising CABIO-P05, CABIO-P07, CABIO-E01, CABIO-E03, CABIO-E04, CABIO-E05, CABIO-E06 and CABIO-E07 as herein described for the first time;
b) comparing, for each locus of the microsatellite loci, the number of indel(s) to a reference number of indel(s) for the locus, and determining the MSI status of said locus, a locus being considered as unstable if the indels count is equal to or above (≥) the reference value for the locus and as stable if the indels count is below (<) the reference value for the locus;
c) calculating a MSID score, ranging between 0 and 1, consisting of the total number of unstable loci on the total number of loci; and
d) determining that the MSI status of the tumor is unstable if the MSID score is equal to or above (≥) 0.5; stable if the MSID score is equal to or below (≤) 0.125, and uncertain if the MSID score is between 0.125 and 0.5; and, optionally
e) if the MSID score is between 0.125 and 0.5, repeating steps a)-d) until the MSI status can be determined.

In particular aspects herein described, the method herein revealed by inventors is for assessing precancerous and/or cancerous cells for microsatellite instability; for detecting a predisposition to develop a cancer; for evaluating the prognostic of a cancer; for monitoring cancer progression or regression; for predicting, evaluating or monitoring the response to a treatment of cancer; for selecting the appropriate treatment of cancer for a subject in need thereof, for selecting patients capable of responding to a treatment of cancer; or for selecting patients for enrolment in a clinical trial for the treatment of cancer.

In a preferred embodiment, the method is a partially or fully computer-implemented method.

Also herein described is a computer-implemented method of training a classifier for determining the microsatellite stability (MSS) or microsatellite instability (MSI) status (also herein identified as “phenotype”) of biological sample, in particular of a tumor, for example of a human tumor, wherein the method comprises:
a) providing a training set of microsatellite loci, each locus being obtained from a DNA sequence of interest, or preprocessed information obtained from said training set, as input to the classifier, said training set comprising i) stable (MSS) microsatellites loci, or sub-sequences thereof, obtained from proficient MisMatch Repair system (pMMR) or MSS cells, DNA, tumors or subjects, known as having a microsatellite stable status or phenotype, and ii) unstable (MSI) microsatellites loci, or sub-sequences thereof, obtained from deficient MisMatch Repair system (dMMR) or MSI cells, DNA, tumors or subjects, known as having a microsatellite unstable status or phenotype;
b) generating an output of the classifier for each microsatellite locus, said output classifying the microsatellite locus input as having stable (MSS) or an unstable (MSI) status or phenotype; and
c) evaluating the classifier's accuracy for distinguishing between a stable (MSS) status or phenotype and an unstable (MSI) status or phenotype by comparing, for each microsatellite locus, the output of the classifier to the known actual phenotype of the microsatellite locus or to a reference number of indels for the microsatellite locus;
wherein the classifier is considered as an accurate classifier to determine the MSS or MSI status or phenotype of a biological sample, in particular of a tumor, if it exhibits an accuracy in counting indels (also known as delins) for each of the microsatellite loci with a resolution of lbp (base pair).

Inventors also herein describe a computing system comprising:
a memory storing at least one instruction of a classifier trained according to a computer-implemented method as herein described, and
a processor accessing to the memory for reading said instruction(s) and executing a method of the invention as herein described.

Also herein described is a kit for analyzing microsatellite loci of genomic DNA, preferably human genomic DNA, comprising a pair of oligonucleotide primers, preferably at least two pairs of oligonucleotide primers, suitable for amplifying or co-amplifying a set of microsatellite loci of human genomic DNA, and/or oligonucleotide probes, preferably at least two oligonucleotide probes, for detecting sequences in said set of microsatellite loci, and optionally a thermostable polymerase and/or control DNA isolated from normal non-cancerous biological material and/or lacking mismatch repair genes. This set typically comprises several, for example from two to eight, preferably at least four, pairs of primers suitable for the amplification of, and/or from two to eight, preferably at least four, oligonucleotide probes for the detection of, a set of markers as herein described, in particular a set comprising at least two microsatellite markers, preferably at least four, for example at least five, six or seven microsatellite markers selected from CABIO-P05, CABIO-P07, CABIO-E01, CABIO-E03, CABIO-E04, CABIO-E05, CABIO-E06 and CABIO-E07, or all of the eight microsatellite markers.

Inventors also herein describe the use of such a kit for the analysis of microsatellite stability or instability.

### BRIEF DESCRIPTION OF THE FIGURES

FIG. 1: Study workflow.

IHC: immunohistochemistry; CRCs: colorectal cancers; ECs: endometrial cancers, MMR: mismatch repair, dMMR: deficient MMR; pMMR: proficient MMR; MSS: microsatellites stability; MSI: microsatellites instability; MSI-PCR: MSI testing by Polymerase Chain Reaction; Octaplex CaBio-MSID: MSI testing by Next Generation Sequencing with MSI Detection tool.

FIG. 2: MSI classification using MSID in CRCs with a conventional 5-markers panel (BAT-25, BAT-26, NR-21, NR-24, MONO-27).

Dot plots show the correlation between pMMR/MSS or dMMR/MSI status initially determined by reference methods (IHC+PCR) and MSI-NGS testing in a retrospective cohort of 303 CRC samples. Following stratification by reference methods 241 pMMR/MSS samples (left) and 62 dMMR/MSI samples (right) were reassessed using MSID, the fraction of unstable microsatellite loci is shown (MSID score values: 0, no unstable marker; 0.20, 1 unstable marker; 0.40, 2 unstable markers; 0.60, 3 unstable markers; 0.80, 4 unstable markers; 1, 5 unstable markers). Microsatellite status was considered stable if MSID score≤0.2, unstable if MSID score≥0.6 and uncertain for MSID score equal to 0.4.

### MSI: Microsatellite Instability; MSS: Microsatellite Stability; MSID: MSI Detection Tool

FIG. 3: Within-laboratory reproducibility of the 8-marker CaBio panel for MSI classification using MSID in FFPE samples.

Dot plots show the reproducibility results obtained for 3 different CRC samples (1 pMMR/MSS and 2 dMMR/MSI) initially stratified by reference methods (IHC+PCR) and reassessed using MSID in 3 independent analytical procedures. The horizontal lines and error bars indicate mean+SD, the fraction of unstable microsatellite loci is shown (MSID score values: 0, no unstable marker; 0.125, 1 unstable marker; 0.25, 2 unstable markers; 0.375, 3 unstable markers; 0.50, 4 unstable markers; 0.625, 5 unstable markers; 0.75, 6 unstable markers; 0.875, 7 unstable markers; 1, 8 unstable markers). Microsatellite status was considered stable if MSID score≤0.125, unstable if MSID score≥0.5 and uncertain for MSID scores equal to 0.25 and 0.375.

MSI: Microsatellite Instability; MSS: Microsatellite Stability; MSID: MSI Detection Tool; MSI-1: MSI-2: Patients with Various Microsatellite Instability Levels

FIG. 4: Overview of microsatellite status determination in a representative selection of FFPE-CRC (A) and FFPE-EC (B) samples.

30 CRCs and 26 ECs were firstly classified by 2 reference techniques: IHC analysis for expression of MMR proteins MLH1/PMS2/MSH2/MSH6 to detect pMMR or dMMR phenotype, and MSI-PCR analysis for determination of MSS or MSI status. Samples were subsequently subjected to blinded analysis of MSS or MSI status by MSI-NGS. MSI-NGS scoring is given for each microsatellite loci (CaBio E01, E03, E04, E05, E06, E07, P05, P07) as MSS (blue) or MSI (red). Samples are grouped according to IHC phenotype and MSI-PCR status.

MSI: Microsatellite Instability; MSS: Microsatellite Stability; MSID: MSI Detection Tool; dMMR: Deficient Mismatch Repair; pMMR: Proficient MisMatch Repair; PCR: Polymerase Chain Reaction; EC: Endometrial Cancer; CRC: Colorectal Cancer; IHC: Immunohistochemistry

FIG. 5: MSI classification using MSID in CRCs with the CaBio octaplex (“CaBio panel”).

Dot plots show the correlation between pMMR/MSS or dMMR/MSI status initially determined by reference methods (IHC+PCR) and MSI-NGS testing in a retrospective cohort of 303 CRC samples. Following stratification by reference methods 241 pMMR/MSS samples (left) and 62 dMMR/MSI samples (right) were reassessed using MSID, the fraction of unstable microsatellite loci is shown (MSID score values: 0, no unstable marker; 0.125, 1 unstable marker; 0.25, 2 unstable markers; 0.375, 3 unstable markers; 0.50, 4 unstable markers; 0.625, 5 unstable markers; 0.75, 6 unstable markers; 0.875, 7 unstable markers; 1, 8 unstable markers). Microsatellite status was considered stable if MSID score≤0.125, unstable if MSID score≥0.5 and uncertain for MSID score equal to 0.25 and 0.375). Comparison of MSID scores obtained for MSI tumors indicates a significant difference relative to MSS tumors (p=2.81.10⁻⁴⁸, Wilcoxon-Mann Withney rank sum test, two sided).

### MSI: Microsatellite Instability; MSS: Microsatellite Stability; MSID: MSI Detection Tool

FIG. 6: Diagnostic performance of CaBio loci in MSI-NGS calling for CRCs versus reference methods (IHC+PCR).

A-B: AUC (Area Under the Curve)-ROC (Receiver Operating Characteristics) curve plots for evaluation of individual loci performance in 41 CRC samples. C: AUC, Sensitivity (Se %) and Specificity (Sp %) calculation for each of the 8 individual loci, 95% confidence intervals (CI) are given.

FIG. 7: MSI classification using MSID in ECs with the CaBio octaplex (“CaBio panel”).

Dot plots show the correlation between pMMR/MSS or dMMR/MSI status initially determined by reference methods (IHC+PCR) and MSI-NGS testing in a retrospective cohort of 88 EC samples. Following stratification by reference methods 60 pMMR/MSS samples (left) and 28 dMMR/MSI samples (right) were reassessed using MSID, the fraction of unstable microsatellite loci is shown (MSID score values: 0, no unstable marker; 0.125, 1 unstable marker; 0.25, 2 unstable markers; 0.375, 3 unstable markers; 0.50, 4 unstable markers; 0.625, 5 unstable markers; 0.75, 6 unstable markers; 0.875, 7 unstable markers; 1, 8 unstable markers). Microsatellite status was considered stable if MSID score≤0.125, unstable if MSID score≥0.5 and uncertain for MSID score equal to 0.25 and 0.375. Comparison of MSID scores obtained for MSI tumors indicate a significant difference relative to MSS tumors (p=2.89.10⁻⁸, Wilcoxon-Mann Withney rank sum test, two sided).

### MSI: Microsatellite Instability; MSS: Microsatellite Stability; MSID: MSI Detection Tool

FIG. 8: Diagnostic performance of CaBio loci in MSI-NGS calling for ECs versus reference methods (IHC+PCR).

A-B: AUC (Area Under the Curve)-ROC (Receiver Operating Characteristics) curve plots for evaluation of individual loci performance in 11 EC samples. C: AUC, Sensitivity (Se %) and Specificity (Sp %) calculation for each of the 8 individual loci, 95% confidence intervals (CI) are given.

### DETAILED DESCRIPTION OF THE INVENTION

In order that the present invention may be more readily understood, certain terms are defined herein. Additional definitions are set forth throughout the detailed description.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art.

The term “amplicon” or “amplicon molecule” refers to a nucleic acid molecule generated by amplification of a template nucleic acid molecule, such as a cfDNA, or a nucleic acid molecule having a sequence complementary thereto, or a double-stranded nucleic acid including any such nucleic acid molecule.

The term “oligonucleotide primer”, or “primer”, refers to a nucleic acid molecule used, capable of being used, or for use in, generating amplicons from a template nucleic acid molecule. Under amplification-permissive conditions (e.g., in the presence of nucleotides and a DNA polymerase, and at a suitable temperature and pH), an oligonucleotide primer can provide a point of initiation of amplification from a template to which the oligonucleotide primer hybridizes. Typically, an oligonucleotide primer is a single-stranded nucleic acid between 5 and 200 nucleotides in length. Those of ordinary skill in the art will appreciate that optimal primer length for generating amplicons from a template nucleic acid molecule can vary with conditions including temperature parameters, primer composition, and amplification method. A pair of oligonucleotide primers, as used herein, refers to a set of two oligonucleotide primers that are respectively complementary to a first strand and a second strand of a template double-stranded nucleic acid molecule. First and second members of a pair of oligonucleotide primers may be referred to as a “forward” oligonucleotide primer and a “reverse” oligonucleotide primer, respectively, with respect to a template nucleic acid strand, in that the forward oligonucleotide primer is capable of hybridizing with a nucleic acid strand complementary to the template nucleic acid strand, the reverse oligonucleotide primer is capable of hybridizing with the template nucleic acid strand, and the position of the forward oligonucleotide primer with respect to the template nucleic acid strand is 5′ of the position of the reverse oligonucleotide primer sequence with respect to the template nucleic acid strand. It will be understood by those of ordinary skill in the art that the identification of a first and second oligonucleotide primer as forward and reverse oligonucleotide primers, respectively, is arbitrary in as much as these identifiers depend upon whether a given nucleic acid strand or its complement is utilized as a template nucleic acid molecule.

The term “probe” refers to a single- or double-stranded nucleic acid molecule that is capable of hybridizing with a complementary target, such as DNA or an amplicon, and includes a detectable moiety. In some instances, e.g., as set forth herein, a probe is a capture probe useful in the detection, identification and/or isolation of a target sequence, such as a gene sequence. In various instances, e.g., as set forth herein, the detectable moiety/fragment of the probe can be, e.g., an enzyme (see ELISA, as well as enzyme-based histochemical assays), a fluorescent moiety, a radioactive moiety, or a moiety associated with a luminescence/light signal.

The “sequence identity” between two sequences is described by the parameter “sequence identity”, “sequence similarity” or “sequence homology”. In the context of the present invention, the “sequence identity” between two sequences (A) and (B) is determined by comparing two sequences aligned in an optimal manner, through a window of comparison. The sequences alignment, or comparison of sequences and determination of percent identity between two sequences, can be accomplished using any methods known in the art. The comparison may for example involve a computational algorithm, such as BLAST (basic local alignment search tool).

The alignment can be carried out by methods well-known in the art, for example, using the Needleman-Wunsch global alignment algorithm, or the Smith-Waterman local alignment algorithm. The analysis software matches similar sequences using similarity measures attributed to various deletions and other modifications. Once the total alignment has been obtained, the identity percentage can be obtained by dividing the total number of identical nucleic acid residues aligned by the total number of nucleic acid residues contained in the longest sequence between the sequences (A) and (B) to compare two nucleic acid sequences, one can use, for example, the BLAST or EMBOSS Needle tool. EMBOSS Needle creates an optimal global alignment of two sequences using the Needleman-Wunsch algorithm.

The term “about”, when used herein in reference to a value, refers to a value that is similar, in context, to the referenced value. In general, one of ordinary skill in the art, familiar with the context, will appreciate the relevant degree of variance encompassed by the term “about” in that context.

Unlike point mutations, which affect only a single nucleotide, microsatellite mutations lead to the gain or loss of an entire repeat unit, and sometimes two or more repeats simultaneously. Thus, the mutation rate at microsatellite loci is expected to differ from other mutation rates, such as base substitution rates. One proposed cause of such length changes is replication slippage, caused by mismatches between DNA strands while being replicated during meiosis. DNA polymerase, the enzyme responsible for reading DNA during replication, can slip while moving along the template strand and continue at the wrong nucleotide. DNA polymerase slippage is more likely to occur when a repetitive sequence (such as CGCGCG) is replicated. Because microsatellites consist of such repetitive sequences, DNA polymerase may make errors at a higher rate in these sequence regions, resulting in alteration in the number of repeats and in the total length of microsatellites. Several studies have found evidence that slippage is the cause of microsatellite mutations (Klintschar M, et al.; Forster P, et al.). Typically, slippage in each microsatellite occurs about once per 1,000 generations (Weber J L, et al.). Thus, slippage changes in repetitive DNA are three orders of magnitude more common than point mutations in other parts of the genome (Jame P, et al.).

As explained in the background section, “microsatellites” and “microsatellite regions”, also called “short tandem repeats” (“STRs”) or “simple sequence repeats” (“SSRs”), designate the repetition, typically from two (2) to fifty (50) times, of a pattern of one or more nucleotides, typically of two (2) to six (6), or up to ten (10) nucleotide motifs, with a minimal length of 5 or 6 bases. The term “tandem” indicates that the repetitions are directly adjacent to each other. Microsatellites occur at numerous loci throughout the genome. The microsatellite sequences are either coding or non-coding sequences. Repeat units of one, two, three, four, five and six nucleotides are referred to as mono-, di-, tri-, tetra-, penta- and hexanucleotide motifs/repeats in a nucleotide sequence, respectively. For example, the sequence TATATATATA is a dinucleotide microsatellite, and GTCGTCGTCGTCGTC is a trinucleotide microsatellite (with “A” being Adenine, “G” being Guanine, “C” being Cytosine, and “T” being Thymine). A particular subclass of microsatellites includes the homopolymers. “Homopolymer” as used herein refers to a microsatellite region that is a mononucleotide repeat of at least 6 (nucleo)bases; in other words, a stretch of at least 6 consecutive guanine (“G”), adenine (“A”), cytosine (“C”) or thymine (“T”) residues/bases if looking at the DNA level. Most particularly, when determining microsatellites, one looks at genomic DNA of a subject (or genomic DNA of a cancer present in the subject).

In the context of the present invention, the terms “microsatellite” and “microsatellite locus” preferably designate mononucleotides repeats or homopolymers, i.e. repetitions of only one of the four bases: A, T, C or G.

In a preferred aspect herein described, the microsatellite is a repeat of “n” thymines (“T”), where “n” designates the number of T and where “n” is for example equal to 20, 21, 22, 23, 24, 25 or 26, preferably equal to 21, 22, 23 or 25.

In a preferred aspect of the invention, the microsatellite locus is selected from the group (also herein identified as “(bio)marker panel” or “(bio)marker set”) comprising, or consisting of, CABIO-P05, CABIO-P07, CABIO-E01, CABIO-E03, CABIO-E04, CABIO-E05, CABIO-E06 and CABIO-E07, said loci being defined with reference to the Homo sapiens (human) reference genome assembly identified as GRCh38 (or hg38) from Genome Reference Consortium human Build 38 patch release 14 (also known as GRCh38.p14) [see NCBI: GCA_000001405.15 GCF_000001405.26]. The person of ordinary skill in the art will understand that by referring to a different reference genome, the location of the herein-described loci may change.

Thus, with reference to the Homo sapiens reference genome assembly identified as GRCh38 or hg38:
CABIO-P05 is a 21 T repeat located at 14q23.1 starting at position chr14: 58359108,
CABIO-P07 is a 21 T repeat located at 7q32 starting at position chr7: 131478596,
CABIO-E01 is a 22 T repeat located at Xq22.3 starting at position chrX: 106849221,
CABIO-E03 is a 23 T repeat located at Xq21.2 starting at position chrX: 85268269,
CABIO-E04 is a 25 T repeat located at 14q32.3 starting at position chr14: 103574079,
CABIO-E05 is a 22 T repeat located at 2p11.2 starting at position chr2: 86456417,
CABIO-E06 is a 23 T repeat located at 4q23 starting at position chr4: 99216136, and
CABIO-E07 is a 21 T repeat located at 20p13 starting at position chr20: 290564.

CABIO-P05 can be amplified for example with the help of a forward primer of SEQ ID NO: 9 and/or of a reverse primer of SEQ ID NO: 10.

CABIO-P07 can be amplified for example with the help of a forward primer of SEQ ID NO: 11 and/or of a reverse primer of SEQ ID NO: 12.

CABIO-E01 can be amplified for example with the help of a forward primer of SEQ ID NO: 13 and/or of a reverse primer of SEQ ID NO: 14.

CABIO-E03 can be amplified for example with the help of a forward primer of SEQ ID NO: 15 and/or of a reverse primer of SEQ ID NO: 16.

CABIO-E04 can be amplified for example with the help of a forward primer of SEQ ID NO: 17 and/or of a reverse primer of SEQ ID NO: 18.

CABIO-E05 can be amplified for example with the help of a forward primer of SEQ ID NO: 19 and/or of a reverse primer of SEQ ID NO: 20.

CABIO-E06 can be amplified for example with the help of a forward primer of SEQ ID NO: 21 and/or of a reverse primer of SEQ ID NO: 22.

CABIO-E07 can be amplified for example with the help of a forward primer of SEQ ID NO: 23 and/or of a reverse primer of SEQ ID NO: 24.

The term “indel” (or “delins”) refers to a mutation class that includes insertion and deletion of a single base or of several bases, as well as the combination thereof. It is classified among small genetic variations, measuring from 1 to 10 000 base pairs in length. An indel inserts or deletes nucleotide(s) from a sequence and thus is distinct from the point mutation occurring in the form of a substitution that replaces one of the nucleotides without changing the overall number of nucleotides in the DNA sequence. In most known genomes, including humans, indel frequency tends to be markedly lower than that of single nucleotide polymorphisms (SNP), except near highly repetitive regions, including homopolymers and microsatellites. In coding regions of the genome, unless the length of an indel is a multiple of 3, it will produce a frameshift mutation. An indel in a microsatellite region results in a net gain or loss of nucleotides.

The presence of an indel can be established by comparing it to DNA in which the indel is not present (e.g. comparing DNA from a tumor sample to germline DNA from the subject with the tumor), or, in case of monomorphic microsatellites or homopolymers, by comparing it to reference genomes. According to specific embodiments, particularly envisaged indels have a length of between 1 and 5 or 6 nucleotides (e.g., the length of the microsatellite or homopolymer is 1 to 5 or 6 nucleotides longer or shorter than the normal known length of the microsatellite or homopolymer). Note that, as indels can be a combination of a insertion and a deletion, the altered nucleic acid sequence may be larger than the length difference (e.g. a deletion of 5 nucleotides combined with an insertion of 3 nucleotides leads to an altered length of 2, but the sequence of the microsatellite may have changed as well). Most typically however, an indel will be either an insertion or a deletion, typically of 1 or 2 nucleotides.

The term “microsatellite status” as used herein can be one of two classes. It refers either to the presence of microsatellite instability (MSI, i.e., a clonal or somatic change in the number of repeated DNA nucleotide units in microsatellites), or to microsatellite stability (MSS, also herein referred to as “absence of MSI”).

Microsatellite instability (MSI) refers to the condition of genetic hypermutability (predisposition to mutation, in particular indel) that may result from impaired DNA mismatch repair (“MMR”), also called deficient DNA mismatch repair (“dMMR”). Considering the correlation between the absence of an intact mismatch repair (MMR) system and the presence of MSI, diagnosing the presence of MSI (or determining MSI status) can be interpreted as diagnosing MMR deficiency. In other words, if microsatellite stability (MSS) reflects or indicates an efficient MisMatch Repair system, MSI is the molecular hallmark of a deficient MMR system.

A cell, DNA, tumor or subject is herein identified as a “MSI cell, DNA, tumor or subject” or as a “dMMR cell, DNA, tumor or subject” (“deficient MisMatch Repair system cell, DNA, tumor or subject”) if the cell, DNA, tumor or subject has a microsatellite unstable phenotype or, in other words if its/the DNA has/exhibits microsatellite instability.

A cell, DNA, tumor or subject is herein identified as a “MSS cell, DNA, tumor or subject” or as a “pMMR cell, DNA, tumor or subject” (“proficient MMR cell, DNA, tumor or subject”) if the cell, DNA, tumor or subject has a microsatellite stable phenotype or, in other words, if its/the DNA does not have/exhibit microsatellite instability.

The most common method to detect MSI is to measure the length of a polymerase chain reaction amplicon containing the entire microsatellite. This typically requires DNA, a pair of primers of which one is often fluorescently end labeled, a sequencer, and suitable software. Alternatively, if the amplicon is sequenced, one can simply count the number of repeat units. MSI can also be indirectly diagnosed by detecting loss of staining by immunohistochemistry (“IHC”) of one of the mismatch repair genes, since this also points to an abnormality in mismatch repair. Immunohistochemical and genetic methods are both characterized by a considerable number of false-negatives, and for this reason combined assessments at the immunohistochemical and genetic level are performed in a routine diagnostic setting.

There are at least 700 000 microsatellites in the human genome. As microsatellite markers were originally quite randomly picked by researchers, based on their own experiments, a conference was held in Bethesda, to discuss the issues and make suggestions to promote consistency across studies. This resulted to the development of guidelines for a “golden standard” marker panel, known as the “Bethesda” panel. This panel consists of three dinucleotide repeats (D2S123, D5S346, D17S250) and two mononucleotide repeats (BAT-26, BAT-25). Recent evolution of MSI detection kits retained BAT-25 and BAT-26 and replaced the 3 dinucleotides markers by more sensitive mononucleotides markers (NR-21, NR-24, MONO-27).

It was proposed to consider a tumor MSI-positive if 40% or more of the markers tested were unstable. When using a five-marker panel, this means that MSI is called when at least two of them are positive; however, often four or all five are positive in tumors with MSI. Tumors that test negative for all five markers are termed microsatellite stable (MSS). Although the initial Bethesda panel and “advanced” Bethesda panels are still considered the standard, it is known to have a fairly low sensitivity (also depending on which MMR gene is mutated). In the field of cancer, another significant disadvantage is that the Bethesda guidelines are specific for colon cancer, even though other cancers displaying MSI are known.

Inventors now herein describe a method of analyzing a set of at least two or at least three microsatellite loci, preferably a set of at least four microsatellite loci, of human DNA, selected from the group comprising:
CABIO-P05, defined as a 21 T repeat located at 14q23.1 and starting at position chr14: 58359108,
CABIO-P07, defined as a 21 T repeat located at 7q32 and starting at position chr7: 131478596,
CABIO-E01, defined as a 22 T repeat located at Xq22.3 and starting at position chrX: 106849221,
CABIO-E03, defined as a 23 T repeat located at Xq21.2 and starting at position chrX: 85268269,
CABIO-E04, defined as a 25 T repeat located at 14q32.3 and starting at position chr14: 103574079,
CABIO-E05, defined as a 22 T repeat located at 2p11.2 and starting at position chr2: 86456417,
CABIO-E06, defined as a 23 T repeat located at 4q23 and starting at position chr4: 99216136, and
CABIO-E07, defined as a 21 T repeat located at 20p13 and starting at position chr20: 290564, with reference to the Homo sapiens (human) reference genome assembly identified as GRCh38 or hg38 from Genome Reference Consortium human Build 38 patch release 14 (also known as GRCh38.p14) [see NCBI: GCA_000001405.15 GCF_000001405.26].

In a particular aspect, the method comprises the analysis of five, six, seven or all of the herein above described eight loci.

The analysis of the nucleic acid sequence, in particular the detection of indel(s) in said sequence, can be carried out by any of the methods well known to those skilled in the art, said method requiring or not an amplification step. Indels can for example be detected by direct sequencing using well-known methods.

As explained herein above, microsatellites are typically analysed by conventional PCR amplification and amplicon size determination (also herein identified as “MSI-PCR”), sometimes followed by DNA sequencing, in particular Sanger sequencing.

The analysis can be performed by extracting nuclear DNA from the cells of a sample of interest, then amplifying specific polymorphic regions of the extracted DNA by means of the polymerase chain reaction (PCR). Once these sequences have been amplified, they are resolved either through gel electrophoresis or capillary electrophoresis, which will allow the analyst to determine how many repeats of the microsatellites sequence in question there are. If the DNA was resolved by gel electrophoresis, the DNA can be visualized for example by silver staining (low sensitivity, safe, inexpensive), or using an intercalating dye such as ethidium bromide (fairly sensitive, moderate health risks, inexpensive), or fluorescent dyes (highly sensitive, safe, expensive). Instruments built to resolve microsatellite fragments by capillary electrophoresis also typically use fluorescent dyes.

Microsatellites can be amplified for identification by the PCR process, using the unique sequences of flanking regions as primers. DNA is repeatedly denatured at a high temperature to separate the double strand, then cooled to allow annealing of primers and the extension of nucleotide sequences through the microsatellite. This process results in production of enough DNA to be visible on agarose or polyacrylamide gels; only small amounts of DNA are needed for amplification because in this way thermocycling creates an exponential increase in the replicated segment (Griffiths, A. J. F., et al.). With the abundance of PCR technology, primers that flank microsatellite loci are simple and quick to use, but the development of correctly functioning primers is often a tedious and costly process.

Multiplex polymerase chain reaction (Multiplex PCR) can be carried out in the context of the invention. Multiplex PCR refers to the use of PCR to amplify several different DNA sequences simultaneously using multiple primers and a temperature-mediated DNA polymerase (as if performing many separate PCR reactions all together within a single reaction). The primer design for all primers pairs/sets as well as the annealing temperatures for each of the primer pairs have to be optimized so that all primers can work at the same annealing temperature during PCR. It is performed within a single PCR mixture to produce amplicons of varying sizes that are specific to different DNA sequences. Amplicon sizes, i.e., their base pair length, should be different enough to form distinct bands when visualized by gel electrophoresis. Alternatively, if amplicon sizes overlap, the different amplicons may be differentiated and visualised using primers that have been dyed for examples with different colour fluorescent dyes. Commercial multiplexing kits for PCR are available and used by many forensic laboratories to amplify degraded DNA samples. By targeting multiple sequences at once, additional information may be gained from a single test run that otherwise would require several times the reagents and more time to perform.

In a preferred aspect, the method of analyzing microsatellite loci of the invention, involves a step of coamplifying a set of microsatellite loci from at least one sample of DNA, preferably in a multiplex amplification reaction, using suitable primers. In particular, the size of the amplified DNA fragments may be determined and/or the amplified DNA fragments may be sequenced. In an advantageous embodiment, the set of microsatellite loci comprises at least two, for example three, four, five, six, seven or eight loci selected from CABIO-P05, CABIO-P07, CABIO-E01, CABIO-E03, CABIO-E04, CABIO-E05, CABIO-E06 and CABIO-E07, preferably at least four, five, six or seven loci of said set, or all of the eight loci of the set.

In a particular aspect, at least one of the primers used in the method has a nucleic acid sequence selected from the group of primer sequences identified by SEQ ID NO: 9-24. In another particular aspect, at least one of the primer pairs used in the method has a pair of sequences selected from SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; and SEQ ID NO: 23 and SEQ ID NO: 24.

In a further particular aspect, the set of loci is co-amplified using a specific oligonucleotide primer pair for each locus of interest, the specific oligonucleotide primers having respectively sequences as set forth in:
SEQ ID NO: 9 and SEQ ID NO: 10, if the locus is CABIO-P05,
SEQ ID NO: 11 and SEQ ID NO: 12, if the locus is CABIO-P07,
SEQ ID NO: 13 and SEQ ID NO: 14, if the locus is CABIO-E01,
SEQ ID NO: 15 and SEQ ID NO: 16, if the locus is CABIO-E03,
SEQ ID NO: 17 and SEQ ID NO: 18, if the locus is CABIO-E04,
SEQ ID NO: 19 and SEQ ID NO: 20, if the locus is CABIO-E05,
SEQ ID NO: 21 and SEQ ID NO: 22, if the locus is CABIO-E06, or
SEQ ID NO: 23 and SEQ ID NO: 24, if the locus is CABIO-E07, or (functionally equivalent, i.e. suitable for amplifying the specific locus) sequences having at least 90% identity thereto (depending on the nature of the targeted locus of interest, said locus being selected from CABIO-P05, CABIO-P07, CABIO-E01, CABIO-E03, CABIO-E04, CABIO-E05, CABIO-E06 and CABIO-E07).

The analysis of microsatellites, in particular of indel(s) in a microsatellite, does not necessarily require use of a complete PCR. In a particular aspect, the method of the invention uses only the extension of a primer by a single, fluorescence-labeled dideoxyribonucleic acid molecule (ddNTP) that is complementary to the nucleotide to be investigated. The nucleotide at the polymorphic site can be identified via detection of a primer that has been extended by one base and is for example fluorescently labeled (e.g., Kobayashi et al., Mol. Cell. Probes, 9:175-182, 1995).

Particular techniques involve using oligonucleotide sequences consisting of repeats complementary to repeats in the microsatellite to “enrich” the DNA extracted (“microsatellite enrichment”). An oligonucleotide probe hybridizes with the repeat in the microsatellite, and the probe/microsatellite complex is then pulled out of solution. The enriched DNA is then cloned as normal (Ostrander et al., Proc. Natl. Acad. Sci. USA, 15; 89(8):3419-23, 1992).

Another way to carry out the analysis of the nucleic acid sequence according to the invention involves the capture of a target nucleic acid sequence of interest with a detectable suitable probe.

In a particular aspect, the allele specific oligonucleotide hybridization (ASO) technique (e.g., Saiki et al., or Stoneking et al.) may be used. This technique relies on distinguishing between two DNA molecules differing by one base by hybridizing an oligonucleotide probe that is specific for one of the variants to an amplified product obtained from amplifying the nucleic acid sample. This method typically employs short oligonucleotides, e.g. 15-30 bases in length. The probes are designed to differentially hybridize to one variant versus another. Principles and guidance for designing such probe is available in the art. Hybridization conditions should be sufficiently stringent that there is a significant difference in hybridization intensity between alleles, and producing an essentially binary response, whereby a probe hybridizes to only one of the alleles. The amount and/or presence of a particular sequence is determined by measuring the amount of sequence-specific oligonucleotide that is hybridized to the sample. Typically, the oligonucleotide is labeled with a label such as a fluorescent label, for detection. For example, a sequence-specific oligonucleotide is applied to immobilized oligonucleotides representing sequences with different microsatellite length. After stringent hybridization and washing conditions, fluorescence intensity is measured for each microsatellite oligonucleotide. Suitable assay formats for detecting hybrids formed between probes and target nucleic acid sequences in a sample are known in the art and include the immobilized target (dot-blot) format and immobilized probe (reverse dot-blot or line-blot) assay formats. In a dot-blot format, amplified target DNA is immobilized on a solid support, such as a nylon membrane. The membrane-target complex is incubated with labeled probe under suitable hybridization conditions, unhybridized probe is removed by washing under suitably stringent conditions, and the membrane is monitored for the presence of bound probe. In the reverse dot-blot (or line-blot) format, the probes are immobilized on a solid support, such as a nylon membrane or a microtiter plate. The target DNA is labeled, typically during amplification by the incorporation of labeled primers. One or both of the primers can be labeled. The membrane-probe complex is incubated with the labeled amplified target DNA under suitable hybridization conditions, unhybridized target DNA is removed by washing under suitably stringent conditions, and the membrane is monitored for the presence of bound target DNA.

In another particular aspect, the nucleic acid sequence analysis method of the invention involves a detectable hybridization probe which is a sequence-specific probe that discriminates between the sequences with and without indels.

In a particular aspect herein described, the probe may have a sequence as set forth in:
SEQ ID NO: 25, if the locus is CABIO-P05,
SEQ ID NO: 26, if the locus is CABIO-P07,
SEQ ID NO: 27, if the locus is CABIO-E01,
SEQ ID NO: 28, if the locus is CABIO-E03,
SEQ ID NO: 29, if the locus is CABIO-E04,
SEQ ID NO: 30, if the locus is CABIO-E05,
SEQ ID NO: 31, if the locus is CABIO-E06, or
SEQ ID NO: 32, if the locus is CABIO-E07, or a (functionally equivalent, i.e. suitable for amplifying the specific locus) sequence having at least 90%, for example 91%, 92%, 93% or 94%, or at least 95%, for example 96%, 97%, 98% or 99% sequence identity thereto (i.e., identity to one of SEQ ID NO: 25-32, depending on the nature of the targeted locus of interest selected from CABIO-P05, CABIO-P07, CABIO-E01, CABIO-E03, CABIO-E04, CABIO-E05, CABIO-E06 and CABIO-E07).

In a particular aspect, the nucleic acid sequence analysis method of the invention uses both a sequence-specific primer and a labeled probe that binds to amplified product.

Amplification products generated using the polymerase chain reaction can be analyzed by the use of denaturing gradient gel electrophoresis. Different sequences (alleles) can be identified based on the different sequence-dependent melting properties and electrophoretic migration of DNA in solution (see, e.g. Erlich, ed., PCR Technology, Principles and Applications for DNA Amplification, W. H. Freeman and Co, New York, 1992, Chapter 7). Distinguishing of microsatellite polymorphisms can be done using capillary electrophoresis. Capillary electrophoresis conveniently allows identification of the number of repeats in a particular microsatellite sequence (allele). The application of capillary electrophoresis to the analysis of DNA polymorphisms is well known to those in the art (see, for example, Szantai, et al, J Chromatogr A. (2005) 1079(1-2):41-9; Bjorheim and Ekstrom, Electrophoresis (2005) 26(13):2520-30 and Mitchelson, Mol Biotechnol. (2003) 24(1):41-68). Alleles of target sequences can be differentiated using single-strand conformation polymorphism analysis, which identifies base differences by alteration in electrophoretic migration of single stranded PCR products, as described, e.g., in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target genes.

Nucleic acid analysis (in particular indel detection) methods often employ labeled oligonucleotides. Oligonucleotides can be labeled by incorporating a label detectable by spectroscopic, photochemical, biochemical, immunochemical, or chemical means. Useful labels include fluorescent dyes, radioactive labels, e.g. 32P, electron-dense reagents, enzyme, such as peroxidase or alkaline phosphatase, biotin, or haptens and proteins for which antisera or monoclonal antibodies are available. Labeling techniques are well known in the art (see, e.g. Sambrook et al.). The microsatellite locus of the herein described marker set/panel can be detected using any of these technologies, or others, the marker panel being independent of the technology used.

According to Tytgat, O. et al., repetitive DNA is not easily analyzed by next generation DNA sequencing (NGS) methods, which struggle with homopolymeric tracts. Therefore, microsatellites are normally analyzed by conventional PCR amplification and amplicon size determination. The use of PCR means that microsatellite length analysis is prone to PCR limitations like any other PCR-amplified DNA locus.

Advantageously, the nucleic acid sequence analysis method of the invention allowing the determination of the microsatellite status, is compatible with NGS (next generation sequencing), also called “massive parallel sequencing”, in particular second generation high-throughput sequencing. Many NGS platforms differ in engineering configurations and sequencing chemistry. They share the technical paradigm of massive parallel sequencing via spatially separated, clonally amplified DNA templates or single DNA molecules in a flow cell. This design is different from that of Sanger sequencing—also known as capillary sequencing or first-generation sequencing—which is based on electrophoretic separation of chain-termination products produced in individual sequencing reactions.

DNA sequencing with commercially available NGS platforms is generally conducted with the following steps. First, DNA sequencing libraries are typically generated by clonal amplification by PCR in vitro. Second, the DNA is sequenced by synthesis, such that the DNA sequence is determined by the addition of nucleotides to the complementary strand rather than through chain-termination chemistry. Third, the spatially segregated, amplified DNA templates are sequenced simultaneously in a massively parallel fashion without the requirement for a physical separation step.

These steps are followed in most NGS platforms, but each utilizes a different strategy.

Two methods are used in preparing DNA templates for NGS reactions: amplified templates originating from single DNA molecules, and single DNA molecule templates. For imaging systems which cannot detect single fluorescence events, amplification of DNA templates is required. The three most common amplification methods are emulsion PCR (emPCR), rolling circle and solid-phase amplification. The final distribution of templates can be spatially random or on a grid.

In emulsion PCR methods, a DNA library is first generated through random fragmentation of genomic DNA. Single-stranded DNA fragments (templates) are attached to the surface of beads with adaptors or linkers, and one bead is attached to a single DNA fragment from the DNA library. The surface of the beads contains oligonucleotides probes with sequences that are complementary to the adaptors binding the DNA fragments. The beads are then compartmentalized into water-oil emulsion droplets. In the aqueous water-oil emulsion, each of the droplets capturing one bead is a PCR microreactor that produces amplified copies of the single DNA template.

Amplification of a population of single DNA molecules by rolling circle amplification in solution is followed by capture on a grid of spots sized to be smaller than the DNAs to be immobilized.

In the context of DNA colony generation, forward and reverse primers are covalently attached at high-density to the slide in a flow cell. The ratio of the primers to the template on the support defines the surface density of the amplified clusters. The flow cell is exposed to reagents for polymerase-based extension, and priming occurs as the free/distal end of a ligated fragment “bridges” to a complementary oligo on the surface. Repeated denaturation and extension results in localized amplification of DNA fragments in millions of separate locations across the flow cell surface. Solid-phase amplification produces up to 20 billion spatially separated template clusters, providing free ends to which a universal sequencing primer is then hybridized to initiate the sequencing reaction.

A similar, but non-clonal, surface amplification method named “bridge amplification”, was adapted for clonal amplification in 1997 by Church and Mitra (1999).

Protocols requiring DNA amplification are often cumbersome to implement and may introduce sequencing errors. The preparation of single-molecule templates is more straightforward protocol which does not require PCR, which can introduce errors in the amplified templates. Single molecule templates are usually immobilized on solid supports using one of at least three different approaches. In the first approach, spatially distributed individual primer molecules are covalently attached to the solid support. The template, which is prepared by randomly fragmenting the starting material into small sizes (for example, −200-250 bp) and adding common adapters to the fragment ends, is then hybridized to the immobilized primer. In the second approach, spatially distributed single-molecule templates are covalently attached to the solid support by priming and extending single-stranded, single-molecule templates from immobilized primers. A common primer is then hybridized to the template. In either approach, DNA polymerase can bind to the immobilized primed template configuration to initiate the NGS reaction. In a third approach, spatially distributed single polymerase molecules are attached to the solid support, to which a primed template molecule is bound. Larger DNA molecules (up to tens of thousands of base pairs) can be used with this technique, and, unlike the first two approaches, the third approach can be used with real-time methods, resulting in potentially longer read lengths.

In a particular aspect, the template preparation method is based on the use of emPCR (or clonal-emPCR), gridded DNA nanoballs (or gridded rolling circle nanoballs), DNA colony generation (or bridge amplification or clonal-bridge amplification), or single molecule.

Sequencing approaches suitable for use in a NGS platform include for example pyrosequencing, reversible dye terminator, oligonucleotide 8-mer chained ligation, oligonucleotide 9-mer chained ligation, native dNTPs proton detection, or phospholinked fluorescent nucleotides.

Pyrosequencing is a non-electrophoretic, bioluminescence method that measures the release of inorganic pyrophosphate by proportionally converting it into visible light using a series of enzymatic reactions. Unlike other sequencing approaches that use modified nucleotides to terminate DNA synthesis, the pyrosequencing method manipulates DNA polymerase by the single addition of a dNTP in limiting amounts. Upon incorporation of the complementary dNTP, DNA polymerase extends the primer and pauses. DNA synthesis is reinitiated following the addition of the next complementary dNTP in the dispensing cycle. The order and intensity of the light peaks are recorded as flowgrams, which reveal the underlying DNA sequence.

The sequencing by reversible terminator chemistry approach uses reversible terminator-bound dNTPs in a cyclic method that comprises nucleotide incorporation, fluorescence imaging and cleavage. A fluorescently-labeled terminator is imaged as each dNTP is added and then cleaved to allow incorporation of the next base. These nucleotides are chemically blocked such that each incorporation is a unique event. An imaging step follows each base incorporation step, then the blocked group is chemically removed to prepare each strand for the next incorporation by DNA polymerase. This series of steps continues for a specific number of cycles, as determined by user-defined instrument settings. The 3′ blocking groups were originally conceived as either enzymatic or chemical reversal. Sequencing by reversible terminator chemistry can be a four-color cycle, or a one-color cycle. “Virtual Terminators”, which are unblocked terminators with a second nucleoside analogue that acts as an inhibitor, may be used. These terminators have the appropriate modifications for terminating or inhibiting groups so that DNA synthesis is terminated after a single base addition.

In the sequencing-by-ligation mediated by ligase enzymes approach, the sequence extension reaction is not carried out by polymerases but rather by DNA ligase and either one-base-encoded probes or two-base-encoded probes. In its simplest form, a fluorescently labelled probe hybridizes to its complementary sequence adjacent to the primed template. DNA ligase is then added to join the dye-labelled probe to the primer. Non-ligated probes are washed away, followed by fluorescence imaging to determine the identity of the ligated probe. The cycle can be repeated either by using cleavable probes to remove the fluorescent dye and regenerate a 5′-PO4 group for subsequent ligation cycles (chained ligation) or by removing and hybridizing a new primer to the template (unchained ligation).

The method of real-time sequencing involves imaging the continuous incorporation of dye-labelled nucleotides during DNA synthesis: single DNA polymerase molecules are attached to the bottom surface of individual zero-mode waveguide detectors (Zmw detectors) that can obtain sequence information while phospholinked nucleotides are being incorporated into the growing primer strand. A unique DNA polymerase which better incorporates phospholinked nucleotides and enables the resequencing of closed circular templates can be used.

Inventors compared the method of the invention with the NGS sequencing of five of the most frequently analyzed microsatellites complying with the Bethesda and ESMO international guidelines. They also compared the NGS sequencing of the microsatellites panels, including both the panels of the invention and the reference panel of the art, to the routine techniques of immunohistochemistry (“IHC”) and “MSI-PCR”, and herein describe results showing the superiority of the method of the invention, and in particular of the herein disclosed panel of up to 8 microsatellite locus, in terms of both sensitivity and specificity.

In a preferred aspect, the method of analyzing microsatellite loci of the invention comprises a step of sequencing a set of microsatellite loci as herein described, preferably (as taught herein above) the set of at least two to eight, even more preferably of at least four, in particular four to eight, microsatellite loci.

In a particular aspect, this method may further comprise a step of co-amplifying the set of microsatellite loci. As appreciated by one in the art, sequence-specific amplification methods can be performed in reaction that employ multiple sequence-specific primers to target particular microsatellite sequences. Primers for such multiplex applications are generally labeled with distinguishable labels or are selected such that the amplification products produced from the alleles are distinguishable by size. Thus, for example, both alleles in a single sample can be identified using a single amplification by gel analysis of the amplification product. As in the case of sequence-specific probes, a sequence-specific oligonucleotide primer may be exactly complementary to one of the polymorphic sequences in the hybridizing region or may have some mismatches at positions other than the 3′-terminus of the oligonucleotide, which mismatches occur at non-polymorphic sites in both nucleotide sequences.

Thus, any primer of SEQ ID NO: 9-24 or probe of SEQ ID NO: 25-32, herein described, or a sequence having at least 90%, for example 91%, 92%, 93% or 94%, or at least 95%, for example 96%, 97%, 98% or 99% sequence identity thereto, as well as any combination thereof, may be advantageously used in the context of such methods.

The description in particular relates to a method, in particular a computer-implemented method, of assessing (or determining) the microsatellite stability (MSS) or microsatellite instability (MSI) status of a tumor. This method comprises the steps of:
a) counting the number of indel(s) in at least two, for example at least three, preferably at least four, five, six, seven or eight microsatellite loci in a sample of DNA, preferably of tumor's DNA, wherein the tumor is preferably a human tumor and the microsatellite loci are preferably selected from the group (/set) comprising CABIO-P05, CABIO-P07, CABIO-E01, CABIO-E03, CABIO-E04, CABIO-E05, CABIO-E06 and CABIO-E07 as herein described;
b) comparing, for each locus of the at least two microsatellite loci, the number of indel(s) to a reference number of indel(s) for the locus, and determining the MSI status of said locus, a locus being considered as unstable if the indels count is equal to or higher than (above) (≥) the reference value for the locus and as stable if the indels count is below (<) the reference value for the locus;
c) calculating a MSID score, ranging between 0 and 1 (limits included), consisting of the total number of unstable loci on the total number of loci; and
d) determining that the MSI status of the tumor is unstable if the MSID score is equal to or higher than (above) (≥) 0.5; stable if the MSID score is equal to or below (≤) 0.125, and uncertain if the MSID score is between 0.125 and 0.5; and, optionally
e) if the MSID score is between 0.125 and 0.5, repeating steps a)-d) until the MSI status can be determined.

In a preferred aspect, the method comprises the amplification of the at least two, for example at least three, preferably at least four microsatellite loci, for example five, six, seven or eight loci. The panel of at least two loci are preferably selected from the group herein disclosed for the first time comprising CABIO-P05, CABIO-P07, CABIO-E01, CABIO-E03, CABIO-E04, CABIO-E05, CABIO-E06 and CABIO-E07.

In a particular aspect of the invention, reference numbers of indel(s) (=instability threshold: mean+1SD) for each of the herein identified locus of interest are provided herein below:
If the locus is CABIO-P05, the reference number of indel(s) is 26;
If the locus is CABIO-P07, the reference number of indel(s) is 31;
If the locus is CABIO-E01, the reference number of indel(s) is 32;
If the locus is CABIO-E03, the reference number of indel(s) is 24;
If the locus is CABIO-E04, the reference number of indel(s) is 28;
If the locus is CABIO-E05, the reference number of indel(s) is 26;
If the locus is CABIO-E06, the reference number of indel(s) is 31; and
If the locus is CABIO-E07, the reference number of indel(s) is 17.

In a particular and preferred aspect of the invention, the disease is a cancer. All or part of the herein described panel of 8 microsatellite loci can be used to assess/detect the microsatellite status in cancer.

The herein described markers can advantageously be used for determining microsatellite (MSI or MSS) status independent of cancer type. Thus, in principle, diagnosing microsatellite status can be done with the markers provided herein for each type of cancer.

The term “cancer” as used herein, refers to different diseases involving unregulated cell growth, also referred to as malignant neoplasm. The term “tumor” is used as a synonym in the application. It is envisaged that this term covers all solid tumor types (carcinoma, sarcoma, blastoma), but it also explicitly encompasses non-solid cancer types such as leukemia, lymphoma or myeloma. Thus, a “sample of tumor DNA” can also be a blood sample from a person with leukemia. Typically, a sample of tumor DNA has at one point been isolated from a subject, particularly a subject with cancer.

Optionally, it has undergone one or more forms of pre-treatment (e.g. lysis, fractionation, separation, purification) in order for the DNA to be sequenced, although it is also envisaged that DNA from an untreated sample is sequenced.

In a particular aspect, the cancer is a mismatch repair (MMR-)deficient tumor or a cancer (known to be associated with microsatellite instability), in particular a tumor or cancer with a high dMMR (deficient MisMatch Repair system) prevalence, i.e. above 15% (>15%), for example a tumour of the gastrointestinal tract, such as a colorectal or gastric tumor, or a tumor of the endometrium. The colorectal cancer may be for example a colon cancer, a cancer of rectosigmoid junction, a rectal cancer, a cancer of anus and/or of anal canal. In another particular aspect, the cancer is a cancer with low dMMR prevalence (<1%), such as prostate cancer and renal cancer.

In a particular aspect, the cancer is an ovarian cancer.

In a preferred aspect, the cancer is a colorectal or an endometrial cancer.

In another aspect, the disease is a condition such as an autosomal dominant genetic condition, for example the Lynch syndrome (also identified as “HNPCC syndrome”), associated by physicians to a high risk of developing colorectal cancer or other cancers including endometrium, ovary, stomach, small intestine, hepatobiliary tract, upper urinary tract, brain, and skin cancer. The increased risk for these cancers is due to inherited mutations that impair DNA mismatch repair.

In a preferred aspect, the tumor's DNA is obtained from a biological sample of a subject having a disease, in particular a cancer, or suspected of having such a disease, and the biological sample is a solid, fluid or semifluid sample, in particular a sample suitable for detecting tumor cells.

In an aspect, the biological sample is a biopsy, particularly a solid or liquid biopsy.

The biological sample may be a solid biopsy. Tissue biopsies require solid matter from the subject's body. This biopsy is generally removed from a solid tumor or from tissues or organs suspecting to comprise tumor cells. Tissue biopsies are generally utilized when a known tumor's location is suspected or confirmed and available for extraction.

The biological sample may otherwise be a liquid biopsy. The liquid biopsy sample is for example a blood, plasma, serum, sputum, bronchial fluid or pleural effusion sample.

In a particular and preferred aspect, the biological sample is a sample comprising, or consisting of, tumor DNA. A “sample of tumor DNA” refers to any sample that can be used as basis for sequencing and wherein DNA from a cancer is present.

Various methods of extracting/isolating nucleic acids, e.g. DNA, from a sample are known in the art. Nucleic acids can be isolated, e.g., without limitation, with a standard DNA purification technique, for example organic extraction or solid phase extraction. DNA extraction may be performed with a kit available in the art such as for example the KAPA Express Extract kit (Kapa Biosystems, Wilmington, MA) or the Maxwell® 16 FFPE LEV DNA purification kit (Promega Corporation, Madison, WI). As, the purification level of extracted (DNA) materials may have an influence on the MSID score, in a preferred aspect of the invention, a known extraction method with higher purity grade is used when performing MSI analysis with a method of the invention. The DNA extraction may be performed for example with the Maxwell® 16 FFPE LEV DNA purification kit (Promega Corporation, Madison, WI) or any other known kits exhibiting similar performances.

As used herein, the term “subject” refers to an individual vertebrate, more particularly an individual mammal, most particularly and preferably an individual human being. A “subject” as used herein is typically a human, but can also be a mammal, particularly domestic animals such as cats, dogs, rabbits, guinea pigs, ferrets, rats, mice, and the like, or farm animals like horses, cows, pigs, goat, sheep, llamas, and the like. A subject can also be a non-mammalian vertebrate, like a fish, reptile, amphibian or bird; in essence any animal which can develop a cancer fulfills the definition.

In a particular aspect, the subject is suffering, or suspected to suffer, from cancer (as herein defined). For example the subject has a MMR-deficient tumor.

In tumour cells, whose controls on replication are damaged, microsatellites may be gained or lost at an especially high frequency during each round of mitosis. Hence a tumor cell line might show a different genetic fingerprint from that of the host tissue, and might present with loss of heterozygosity.

In another particular aspect, the subject is a patient having a tumor who has been identified as sensitive to a particular therapeutic agent or therapy, as determined by the microsatellite status of the tumor.

In particular aspects herein described, the method herein revealed by inventors is for assessing precancerous and/or cancerous cells for microsatellite instability; for detecting a predisposition to develop a cancer; for evaluating the prognostic of a cancer; for monitoring cancer progression or regression; for predicting, evaluating or monitoring the response to a treatment of cancer; for selecting the appropriate treatment of cancer for a subject in need thereof, for selecting patients capable of responding to a treatment of cancer; or for selecting patients for enrolment in a clinical trial for the treatment of cancer.

This method typically comprises a step of analyzing a set of at least four microsatellite loci of human DNA selected from the group comprising CABIO-P05, CABIO-P07, CABIO-E01, CABIO-E03, CABIO-E04, CABIO-E05, CABIO-E06 and CABIO-E07, as described herein above for the first time. In a preferred aspect, the method is a method of assessing the microsatellite stability (MSS) or microsatellite instability (MSI) status of a tumor as described herein above for the first time. This method preferably comprises the steps of:
a) counting the number of indel(s) in at least four microsatellite loci in a sample of tumor's DNA, wherein the microsatellite loci are selected from the group comprising CABIO-P05, CABIO-P07, CABIO-E01, CABIO-E03, CABIO-E04, CABIO-E05, CABIO-E06 and CABIO-E07 as herein described;
b) comparing, for each locus of the at least four microsatellite loci, the number of indel(s) to a reference number of indel(s) for the locus, and determining the MSI status of said locus, a locus being considered as unstable if the indels count is ≥the reference value for the locus and as stable if the indels count is <the reference value for the locus;
c) calculating a MSID score, ranging between 0 and 1, consisting of the total number of unstable loci on the total number of loci; and
d) determining that the MSI status of the tumor is unstable if the MSID score is ≥0.5; stable if the MSID score is ≤0.125, and uncertain if the MSID score is between 0.125 and 0.5; and, optionally
e) if the MSID score is between 0.125 and 0.5, repeating steps a)-d) until the MSI status can be determined.

As used herein, the terms “diagnosis” and “diagnosing” refer to determining whether (and/or the qualitative or quantitative probability/likelihood that) a subject has or will develop a disease, disorder, condition, or state. For example, in diagnosing a cancer, the diagnosis can include a determination of the risk, type, stage, malignancy, or other classification of a cancer. In some instances, e.g., as set forth herein, a diagnosis can be, or include, determining the prognosis and/or likely response to one or more general or particular therapeutic agents or regimens.

“Diagnosing the microsatellite status of a tumor”, “diagnosing the microsatellite MSI or MSS status of a tumor”, or “diagnosing the microsatellite status of a tumor in a subject”, are all considered synonyms herein. Determining (or diagnosing) the microsatellite status typically implies drawing the conclusion of MSI or MSS based on detecting the presence of one or more indels in the microsatellite regions under investigation, or the conclusion of absence of microsatellite instability based on not detecting indels in the microsatellite regions under investigation. Accordingly, “determining the presence of an indel” in a microsatellite region means assessing or detecting the presence or absence of an indel in said microsatellite region. Likewise, determining the presence of an indel in at least two microsatellite regions means assessing or detecting the presence or absence of an indel in each of said at least two microsatellite regions.

The term “treatment” refers to any act intended to ameliorate the health status of patients such as therapy, prevention, prophylaxis and retardation of the disease or of the symptoms of the disease. It designates both a curative treatment and/or a prophylactic treatment of the disease. A curative treatment is defined as a treatment resulting in cure or a treatment alleviating, improving and/or eliminating, reducing and/or stabilizing a disease or the symptoms of a disease or the suffering that it causes directly or indirectly. A prophylactic treatment comprises both a treatment resulting in the prevention of a disease and a treatment reducing and/or delaying the progression and/or the incidence of a disease or the risk of its occurrence. In certain aspects, such a term refers to the improvement or eradication of a disease, a disorder, an infection or symptoms associated with it. In other aspects, this term refers to minimizing the spread or the worsening of the disease. Treatments according to the present invention do not necessarily imply 100% or complete treatment. Rather, there are varying degrees of treatment recognized by one of ordinary skill in the art as having a potential benefit or therapeutic effect. Preferably, the term “treatment” refers to the application or administration of a composition including one or more active agents to a subject who has a disorder/disease.

In a particular aspect, the treatment is cancer treatment. Preferably, the anti-cancer treatment is selected from the group consisting of resection surgery, chemotherapy, radiotherapy or immunotherapy. Preferably, the therapeutic compound is a chemotherapeutic or immunotherapeutic compound. Chemotherapeutic compounds may be, without limitation, alkylating agents, antimetabolites, plant alkaloids, topoisomerase inhibitors, and antitumor antibiotics. Immunotherapeutic compounds may be for example and without limitation, antibodies, cytokines or interferons.

In a particular aspect herein described, the method herein revealed by inventors is for detecting or identifying tumors with MMR deficiency, predicting, evaluating or monitoring the response to a treatment of cancer, or for selecting patients capable of responding to a treatment of cancer.

Published work for example suggests that mismatch repair (MMR-)deficient tumor or cancer known to be associated with microsatellite instability (as described herein) have a distinct response to standard treatments, typically standard chemotherapy such as 5-fluoracil used in colorectal cancer, or cisplatin and carboplatin used in endometrial cancer, and the alkylating agents such as temozolomide, and emerging targeted therapies. Preclinical investigations suggest, for instance, these familial dMMR cancer patients show higher sensitivity to anti-EGFR (e.g. gefitinib, erlotinib, cetuximab, panitumumab) and VEGF targeted therapies. The precise reason for this heterogeneity is unknown, but the determination of the microsatellite (MSI or MSS) status, or in other words the presence or absence of mutations as a consequence of MMR-deficiency, is critical in determining treatment outcome.

This method preferably includes a step of assessing the microsatellite stability (MSS) or microsatellite instability (MSI) status of a tumor involving the analyzes of a set of at least two, preferably at least four microsatellite loci of human DNA selected from the group comprising CABIO-P05, CABIO-P07, CABIO-E01, CABIO-E03, CABIO-E04, CABIO-E05, CABIO-E06 and CABIO-E07, as described herein above for the first time.

In specific cases, the method preferably includes an additional step of optimizing therapy based on said microsatellite status (i.e. based on whether the cancerous tumor was found to be MSI or MSS), by determining in particular that if a tumor is identified as a MSS tumor, said tumor does not, if the tumor has been previously exposed to a particular therapeutic agent/therapy, or will not, if it has never been previously exposed to a particular therapeutic agent/therapy, respond to the particular therapeutic agent/therapy, and on the contrary that if a tumor is identified as a MSI tumor, said tumor does, if the tumor has been previously exposed to a particular therapeutic agent/therapy, or will, if it has never been previously exposed to a particular therapeutic agent/therapy, respond to the particular therapeutic agent/therapy, i.e. the cancer cells will die, stop growing or proliferate less when treated with such.

In a particular aspect, the method also preferably includes an additional step for predicting response to an immune checkpoint inhibitor (“ICI”, e.g. Pembrolizumab, Dostarlimab, and similar compounds), the detection of a MSI or dMMR being predictive of (clinical) response to ICI whereas the detection of a MSS or pMMR being predictive of an absence of (clinical) response, for example of resistance, to ICI. In certain cancer types such as, in particular, EC, CRC, cholangiocarcinoma, and pancreatic cancer, this additional step for predicting response to ICI is preferably performed before any therapeutic treatment of the subject suffering of cancer [Andre T et al., Berton D et al., Marabelle A et al.].

In another particular aspect, the method also includes an additional step of selecting the appropriate treatment of cancer for a subject in need thereof based on said microsatellite status (i.e. based on whether the cancerous tumor was found to be MSI or MSS). For example, in this regard, adjuvant chemotherapy with fluoropyrimidine plus oxaliplatin in MMR-deficient CRCs improves disease-free survival compared with systemic fluoropyrimidine treatment alone [Tougeron et al.].

Selecting the most appropriate (optimized) treatment for a given subject (patient), i.e., the therapeutic product to which a MSS or MSI tumor is sensitive even if it is resistant to the standard treatment, is a way to limit, preferably overcome, resistance that may be observed when using commonly used therapies such as targeted therapy.

In another particular aspect, the method further comprises a step of treating the subject, preferably with the appropriate or optimized treatment of cancer.

In these methods, the cancer is preferably selected from a cancer of the gastrointestinal tract, such as a colorectal or gastric cancer, or a cancer of the endometrium.

Also herein disclosed is a method for generating a personalized cancer treatment report, the method comprising: acquiring a sample from a subject having or suspected of having cancer, determining the microsatellite status of the tumor with a method as herein described for the first time by inventors, and generating a personalized cancer treatment report to memorize the presence or absence of microsatellite instability in the subject (i.e., in the tumor of the subject).

In a particular aspect, the cancer treatment report comprises one or more of the following: (i) suspicion of genetic predisposition, (ii) information on prognosis, resistance to treatment, or potential therapeutic options; (iii) information on the likely effectiveness of a therapeutic option; (iv) the acceptability of a therapeutic option, or the advisability of applying the therapeutic option to the subject; or (v) information on the administration of a drug.

Further herein provided is a method of screening sensitivity or resistance of cancer cells to a particular treatment, in particular to treatment with a test compound, comprising determining the microsatellite status in the cancer cells using the method herein disclosed by inventors for the first time and associating said status to sensitivity or to resistance of the cancer cells to the treatment, in particular to the test compound. According to a particular aspect, the screening of sensitivity or resistance is used in selecting an appropriate treatment of cancer for a subject suffering of cancer, or in stratifying or classifying the subject for a clinical trial.

Although the herein described methods can in principle be performed in vivo, ex vivo and in vitro, it is particularly envisaged that they are performed in vitro.

In a preferred embodiment, the method of the invention is a partially or fully computer-implemented method.

The term “computer-implemented method” refers to a method which involves a programmable apparatus/device, in particular a computer, computer network, or readable medium carrying a computer program, in which at least one step of the method is performed by using at least one computer program. A computer-implemented method may further comprise at least one step that is not performed by using a computer program.

In a particular and preferred aspect, the method of the invention makes it possible to distinguish, for a particular tumor, a MSS status/profile from a MSI status/profile.

In another particular aspect, the method of the invention makes it possible to distinguish, for a particular biological sample (in particular a tumor sample) from a subject, a healthy or diseased (in particular cancerous) status/profile of the biological sample (and thus of the subject), on the basis of the MSS or MSI status/profile of said biological sample.

Also herein described is a computer-implemented method of training a classifier for (accurately) determining the microsatellite stability (MSS) or microsatellite instability (MSI) status (also herein identified as “phenotype”) of a biological sample, in particular of a tumor, for example of a human tumor, wherein the method comprises:
a) providing a training set of microsatellite loci, each locus being obtained from a DNA sequence of interest, or preprocessed information obtained from said training set, as input to the classifier, said training set comprising i) stable (MSS) microsatellites loci, or sub-sequences thereof, obtained from proficient MisMatch Repair system (pMMR) or MSS cells, DNA, tumors or subjects, known as having a microsatellite stable status or phenotype, and ii) unstable (MSI) microsatellites loci, or sub-sequences thereof, obtained from deficient MisMatch Repair system (dMMR) or MSI cells, DNA, tumors or subjects, known as having a microsatellite unstable status or phenotype, in particular tumor cells;
b) generating an output of the classifier for each microsatellite locus, said output classifying the microsatellite locus input as having a stable (MSS) or an unstable (MSI) status or phenotype; and
c) evaluating the classifier's accuracy for distinguishing between a stable (MSS) status or phenotype and an unstable (MSI) status or phenotype by comparing, for each microsatellite locus, the output of the classifier to the known actual phenotype of the microsatellite locus or to a reference number of indels for the microsatellite locus;
wherein the classifier is considered as an accurate classifier to determine the MSS or MSI status or phenotype of biological sample, in particular of a tumor, if it exhibits an accuracy in counting indels (also known as delins) for each of the microsatellite loci with a resolution (or instability threshold) of lbp (base pair). The instability threshold is calculated as the mean+1SD (Standard Deviation) of indels count in a particular MSS training set of microsatellite loci.

As used herein, the term “classifier” refers to an algorithm that implements classification, i.e. that can determine a likelihood score or a probability that an object classifies within a group of objects (e.g., a group of MSS profiles) as opposed to one or several other groups of objects (e.g., a group of MSI profiles), and that maps said input object to a category (e.g. benign or malignant profiles). The term “classifier” may refer to one or multiple classifiers. For example, multiple classifiers may be trained, which may process data in parallel and/or as a pipeline. For example, output of one type of classifier (e.g., from intermediate layers of a neural network) may be fed as input into another type of classifier.

Examples of classifiers that can be used in the context of the present invention include for example, but are not limited to, neural networks of various architectures (e.g., artificial, deep, convolutional, fully connected) and supervised machine learning classifiers such as Support Vector Machine (SVM) classifier, random forest classifier, decision tree classifier, K-nearest neighbor classifier (KNN), logistic regression classifier, nearest neighbor classifier, Gaussian mixture model (GMM), nearest centroid classifier and linear regression classifier. It is not an exhaustive list and the skilled person in the art will be able to identify similar algorithms that can be equally used, although they are not specifically mentioned here. Details and rules of functioning of the mentioned algorithms have already been widely described in the literature. An important contribution is the set of input data provided to the classifier (i.e. a set of nucleic acid sequences comprising microsatellites or preprocessed information obtained from a set of such nucleic acid sequences). Based on this input data, it is possible to create a suitable model using any appropriate supervised or unsupervised machine learning techniques. The selection of appropriate algorithms is therefore of secondary nature and can be carried out in many different ways and in various combinations obvious to those skilled in the art.

Preferably, the classifier is selected from random forest (RF) classifier, Support Vector Machine (SVM) classifier, decision tree classifier, K-nearest neighbor classifier (KNN), logistic regression classifier, nearest neighbor classifier, Gaussian mixture model (GMM) classifier, nearest centroid classifier, linear regression classifier, and a neural network such as an artificial, deep, convolutional or fully connected neural network. More preferably, the classifier is selected from Support Vector Machine (SVM) classifier, random forest (RF) classifier and neural networks, in particular convolutional neural network (CNN). Even more preferably, the classifier is random forest classifier.

A classifier utilizes some training data to understand how given input objects belong to a category/class or another. The classifier may be provided with a training set of biological samples from subjects, such as pMMR/MSS and/or dMMR/MSI subjects, in particular cancerous subjects (typically cancerous dMMR/MSI subjects), said biological samples comprising, or consisting of, DNA sequences, in particular DNA sequences comprising, or consisting of, microsatellite loci, in particular (benign and/or malignant) tumor DNA. In a preferred aspect, the microsatellite loci are from human DNA and selected from the group comprising CABIO-P05, CABIO-P07, CABIO-E01, CABIO-E03, CABIO-E04, CABIO-E05, CABIO-E06 and CABIO-E07, as described herein above for the first time.

In a preferred embodiment, the unstable (MSI), or stable (MSS), microsatellites loci used to prepare the training set(s) are obtained from the DNA of a cancerous tumor cell, the cancer being any cancer as herein above identified, such as colorectal cancer or endometrial cancer. Alternatively, the classifier may be provided with preprocessed information obtained from such a training set of DNA sequences.

The accuracy of the classifier may be assessed using any method known by the skilled person. In particular, the classifier's accuracy may be assessed by calculating for each of the microsatellite loci the inherent instability threshold (also known as “Peak threshold” in the MSID software). The instability threshold is a measure that evaluates for each of the loci the empirical cut-off value and is defined as: the mean+1SD (standard deviation) of indels count in the MSS training set of microsatellite loci.

The classifier is considered an accurate classifier if it provides instability values equal to or above (≥) lbp relative to the instability threshold at each of the loci for specimens known as being unstable (/ biological samples). This result depicts a perfect classification, i.e., indicates that the two categories, pMMR/MSS and dMMR/MSI are completely distinguishable. An instability value equal to or above (≥) lbp at the instability threshold is considered to allow a good separation between pMMR/MSS and dMMR/MSI phenotypes (/status/profiles/conditions).

The classifier is considered an accurate classifier if it provides instability values (strictly) below (<) 1 bp relative to the instability threshold at each of the loci for stable specimens.

If the classifier exhibits an insufficient accuracy, the training method, i.e. steps a) to c), may be reiterated, in particular with some modifications such as by increasing the number of pMMR or MSS and/or dMMR or MSI microsatellite profiles in the training set of DNA sequences, by using a distinct training set of DNA sequences, for example by modifying some parameters of the classifier, until achieving a satisfying accuracy, as defined herein above.

The pMMR or MSS and/or dMMR or MSI microsatellite profiles may be obtained from biological samples/specimens (such as samples of cells, tumors, subjects, etc., in particular cancerous ones). Preferably, the microsatellite profiles of a DNA sequence of interest, or of sub-sequences thereof, is determined with a method for determining a microsatellite profile as disclosed hereinabove.

Thus, a possibility of increasing the classifier's accuracy is to increase the number of sets of subsequences of DNA of interest used, in order to reach instability values≥lbp relative to instability thresholds.

The training set of microsatellite loci must include a set of at least 20 non-repetitive pMMR/MSS microsatellite profiles (each microsatellite locus having a known pMMR/MSS profile) and a set of at least 20 non-repetitive dMMR/MSI microsatellite profiles (each microsatellite locus having a known dMMR/MSI profile). Said sets are sets which have been previously validated by means of a methodological independent approach known by the skilled person such as immunohistochemistry and/or PCR-based fragment sizing assay.

The evaluation of the classifier's accuracy carried out in step c) of the herein above described method is preferably based on the classification into a MSS or MSI status of the microsatellite locus with a test set comprising microsatellite loci obtained from pMMR/MSS cells and microsatellite loci from dMMR/MSI cells, preferably cancerous cells, said test set being distinct from the training set, the stable (MSS) or unstable (MSI) status of each microsatellite locus being known, and the microsatellite status of each locus of the test set being obtained and processed using the same method as that used to obtain and process the microsatellite status of each locus with the training set.

In a preferred embodiment, the method of the invention uses a classifier trained to determine the microsatellite stable (MSS) or unstable (MSI) profile, status, condition or phenotype of a tumor as herein described.

In a particular embodiment, the invention concerns an in vitro method of determining the microsatellite profile, status, condition or phenotype of a tumor, wherein the method comprises the steps of:
(i) providing a DNA sequence of interest from the tumor of a subject, or preprocessed information obtained from said DNA sequence, said DNA sequence of interest being a DNA sequence comprising microsatellites, in particular at least two (preferably at least four, five, six, seven or eight) microsatellite locus selected from the group/panel comprising, or consisting of, CABIO-P05, CABIO-P07, CABIO-E01, CABIO-E03, CABIO-E04, CABIO-E05, CABIO-E06 and CABIO-E07, as an input to a classifier trained to distinguish between a MSS and MSI profile, status, condition or phenotype, and (ii) using the classifier to identify the microsatellite profile, status, condition or phenotype of the DNA sequence of interest of the tumor of the subject as a MSS profile if the MSID score is equal to or below ≤0.125, or as a MSI profile if the MSID score is equal to or above ≥0.5, as an output of the classifier.

The MisMatch Repair system (MMR), encoded by MLH1, MSH2, PMS2 and MSH6 genes, is involved in repairing these errors [Evrard et al.]. In a preferred embodiment, the method of the invention further comprises a step of assessing the expression, or loss of expression, of at least one of the MLH1, MSH2, MSH6 and PMS2 proteins of the Mismatch Repair System (MMR) system.

In another preferred embodiment, the method involves Next-Generation Sequencing (NGS) and further optionally comprises assessing the expression, or loss of expression, of at least one of the MLH1, MSH2, MSH6 and PMS2 proteins of the Mismatch Repair (MMR) system.

The method of determining if a subject's cancer displays microsatellite instability according to the invention may be performed once or several time during a subject's lifetime. Thus, it is possible to monitor the occurrence of microsatellite instability and the evolution of microsatellite instability.

In a particular embodiment, the subject suffering from cancer is a subject having received/been exposed to an anti-cancer treatment such as resection surgery, chemotherapy, radiotherapy or immunotherapy.

In a particular aspect, the DNA from the subject or from a tumor of the subject is provided once to determine if the subject's cancer displays microsatellite instability, for detecting a predisposition to develop a cancer, for evaluating the prognostic of a cancer, for monitoring cancer progression or regression; for predicting, evaluating or monitoring the response to a treatment of cancer, for selecting the appropriate treatment of a cancer, for selecting patients capable of responding to a treatment of cancer, for selecting patients for enrolment in a clinical trial for the treatment of cancer.

The methods of determining if a subject suffers from cancer according to the invention may also be performed after a first line of treatment, e.g., six months, one year, two years, three years, four years, five years, or ten years after the first line of treatment for monitoring cancer progression or regression.

The DNA from the subject may be provided once or several times during a second line of treatment.

The efficiency of the first and/or second lines of treatment may be assessed by a method of monitoring the response to an anti-cancer treatment, in particular to a therapeutic compound. Such method may be performed once or several times during the first, second and/or later line of treatment.

Inventors also herein describe a computing system comprising:
a memory storing at least one instruction of a classifier trained according to a computer-implemented method as herein described, in particular a method of training a classifier for accurately distinguishing between a pMMR/MSS microsatellite profile and a dMMR/MSI microsatellite profile, and
a processor accessing to the memory for reading said instruction(s) and executing a method of the invention as herein described, in particular a method for assessing precancerous and/or cancerous cells for microsatellite instability, for detecting a predisposition to develop a cancer; for evaluating the prognostic of a cancer; for monitoring cancer progression or regression; for predicting, evaluating or monitoring the response to a treatment of cancer; for selecting the appropriate treatment of cancer for a subject in need thereof; for selecting patients capable of responding to a treatment of cancer; or for selecting patients for enrolment in a clinical trial for the treatment of cancer.

Also herein described is a kit for analyzing microsatellite loci of genomic DNA, preferably human genomic DNA, comprising the tools to genotype at least two, for example three, preferably at least four, five, six, seven or the eight microsatellite markers of the set. Thus, the kit may comprise oligonucleotide primers for co-amplifying such a set of microsatellite loci of human DNA, and/or oligonucleotide probes for detecting particular sequences in the microsatellite loci of the set, in particular indels, and optionally a thermostable polymerase and/or control DNA isolated from normal non-cancerous biological material and/or lacking mismatch repair genes.

The kit typically comprises several, for example from two to eight, for example three, preferably at least four, five, six, seven or eight pairs of primers suitable for the amplification of, and/or several, for example from two to eight, for example three, preferably at least four, five, six, seven or eight oligonucleotide probes for the detection of, a set of markers as herein described, in particular a set comprising at least two, for example three, preferably at least four, five, six, seven or the eight microsatellite markers herein identified as CABIO-P05, CABIO-P07, CABIO-E01, CABIO-E03, CABIO-E04, CABIO-E05, CABIO-E06 and CABIO-E07.

Inventors also herein describe the use of such a kit for the analysis of microsatellite stability or instability.

In a particular aspect, at least one, preferably at least two, oligonucleotide primer(s) or probe(s) for several of (at least two, for example three, four, five, six or seven), or each of, the several microsatellite loci of the set/panel herein described for the first time is fluorescently labelled.

The invention also concerns the use of a kit according to the invention, for amplifying all or part of microsatellite region(s) herein described, preferably CABIO-P05, CABIO-P07, CABIO-E01, CABIO-E03, CABIO-E04, CABIO-E05, CABIO-E06 and/or CABIO-E07, in particular by PCR multiplex, preferably in the context of NGS, for example for the determination of the microsatellite (MSS or MSI) status.

The following examples are provided to better illustrate particular aspects and advantages of the present invention. They should be regarded as illustrative only and not limiting. The application is limited only by the claims.

### Examples

### Materials & Methods

### Study Specimens

In this study, colorectal cancer (CRC) and endometrial cancer (EC) samples were collected from patients who underwent microsatellite evaluation at the Poitiers University Hospital in daily practice.

DNA extraction was performed with the KAPA Express Extract kit (Kapa Biosystems, Wilmington, MA) or the Maxwell® 16 FFPE LEV DNA purification kit (Promega Corporation, Madison, WI). All samples used in this study underwent routine analysis for pMMR/dMMR phenotype using IHC with four MMR proteins (MSH2, MSH6, PMS2, and MLH1 with following antibodies:anti-MSH2 (clone G219-1129 Ventana®, kit Optiview® for revelation), anti-MSH6 (clone 44BD Biosciences® San Jose, USA®, kit Ultraview® for revelation), anti-PMS2 (clone EPR 3947 Ventana®, ready for use; kit Optiview® for revelation with amplification) and anti-MLH1 (clone M1 Ventana® Tucson, USA®, kit Optiview® Tucson, USA® for revelation), and for MSS/MSI status using MSI-PCR method (Promega MSI Analysis System, Version 1.2, Promega Corporation, Madison, WI) as previously described [Guyot D′Asnières De Salins A et al.].

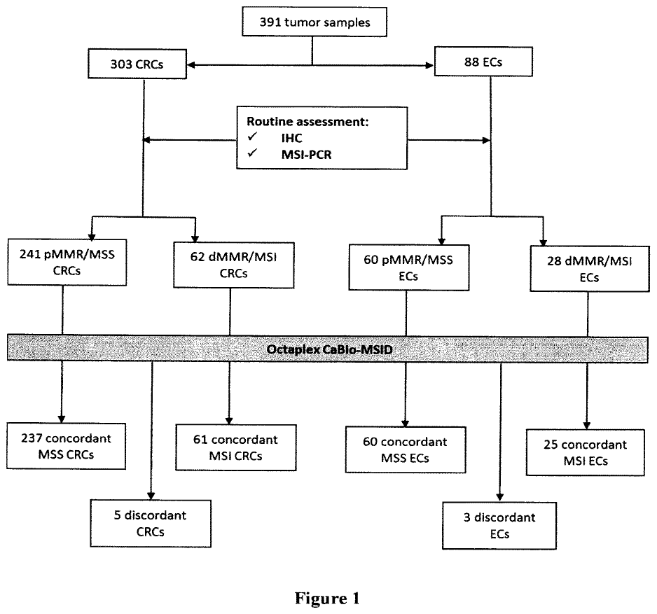

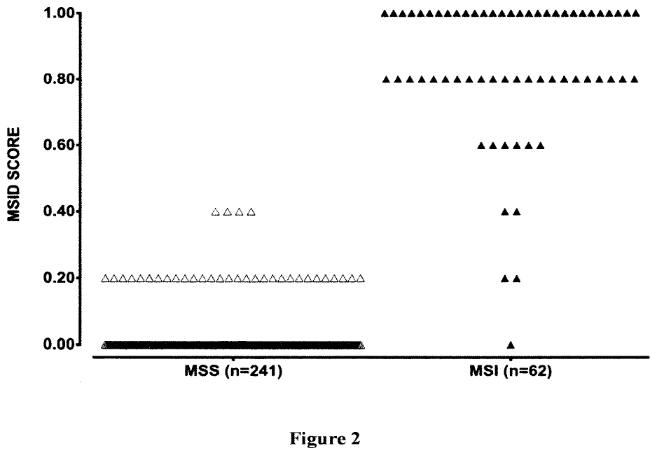

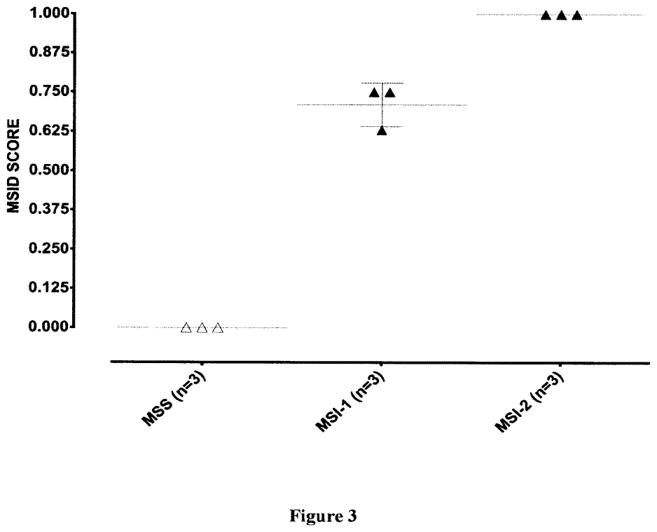

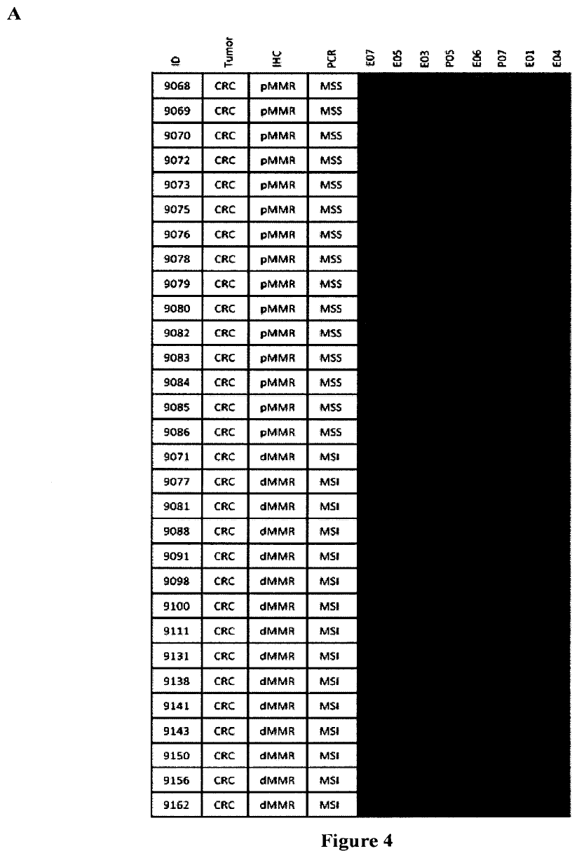

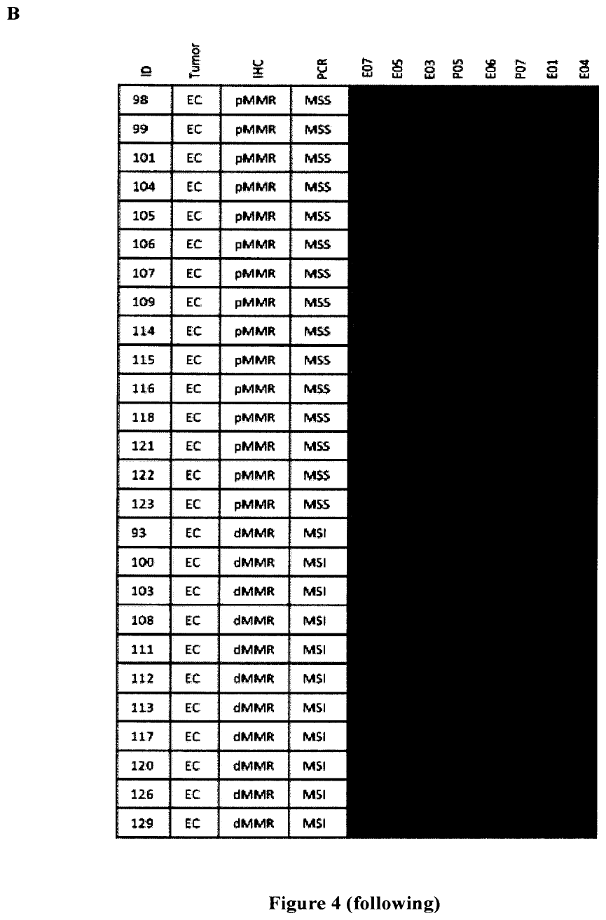

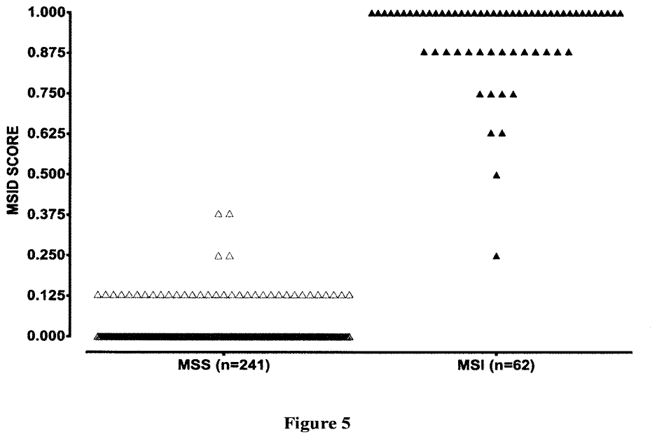

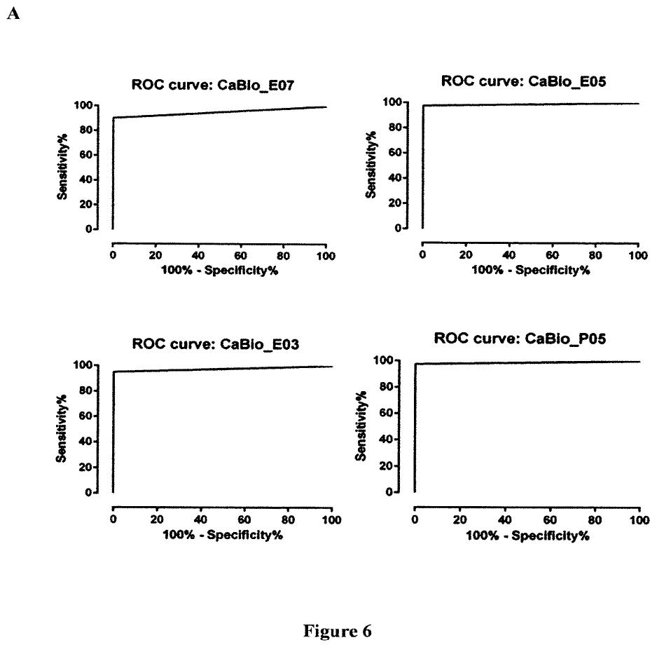

MSI-NGS analysis was performed with a in house designed 8-marker panel (“CaBio”) or 5-marker panel (for CRC only) (FIG. 1). These assays were carried out as blind studies.

### Microsatellite Markers Screening

Previous reports have shown that mononucleotide repeats were the most sensitive markers for MSI detection in humans, with 96% mutation rate in MMR-deficient tumors [Buhard O et al., Bacher J W et al. (2005), Bacher J W et al. (2004), Dietmaier W et al.]. Based on this evidence, inventors carried out investigations for a panel of mononucleotide markers compliant with NGS sequencing which could prospectively be applied to the determination of MSI status in colorectal and endometrial tumors. Retrieval from MSDB v3.0.2 database (MicroSatellite Database, CSIR-Centre for Cellular and Molecular Biology, Uppal Road, Hyderabad—500007, Telangana, India) allowed the identification of 706,849 Simple Sequence Repeats (SSRs) [Avvaru A K et al.]. After the screening process, 13 SSR motifs were retained with enhanced operability for NGS sequencing. Using computational processing inventors identified in particular an octaplex (8 markers) panel, which was named “CaBio octaplex” (also herein identified as “CaBio”) (Table 1), showing a very advantageous level of sensitivity and specificity compatible with routine diagnostic. The tests were reproduced with success by inventors using respectively only 7, 6, 5 and finally 4 markers arbitrarily chosen from the original set of 8 markers.

TABLE 1
Details of the 8 microsatellite markers from CaBio panel

| Marker | Locus | Repeat motif | Start | End |
|---|---|---|---|---|
| CaBio-P05 | 14q23.1 | (T)21 | 58359108 | 58359128 |
| CaBio-P07 | 7q32 | (T)21 | 131478596 | 131478616 |
| CaBio-E01 | Xq22.3 | (T)22 | 106849221 | 106849242 |
| CaBio-E03 | Xq21.2 | (T)23 | 85268269 | 85268291 |
| CaBio-E04 | 14q32.3 | (T)25 | 103574079 | 103574103 |
| CaBio-E05 | 2p11.2 | (T)22 | 86456417 | 86456438 |
| CaBio-E06 | 4q23 | (T)23 | 99216136 | 99216158 |
| CaBio-E07 | 20p13 | (T)21 | 290564 | 290584 |

### Molecular Screening of Microsatellite Loci

Amplification of the mononucleotide repeat markers was performed using the Fluidigm Access Array™ System (Fluidigm Corporation, South San Francisco, CA, USA). Access array-based PCR amplification of specific microsatellite markers (target enrichment) was performed on LP 48.48 IFC according to manufacturer instructions, allowing parallel amplification of 48 samples. Samples were barcoded and tagged with adapter sequences during the target enrichment step allowing for multiplex sequencing. Next generation sequencing was carried out using the Illumina NextSeq 550 system (Illumina, San Diego, CA, USA). The panel includes 29 genes (71 exons) with the genes KRAS, NRAS, BRAF, POLE, and PIK3CA. New 8 markers were included in this panel. MSID algorithm was used to analyse the dataset and for classification of microsatellite instability.

### MSID Algorithm and MSI Classification

Sequencing reads obtained from each sample were aligned on human genome GRCh37 (hg19) with BWA software, version 0.7.17 [Li H, Durbin R (2009)]. The alignment process was performed with the MEM algorithm of BWA using default options. The generated BAM files were processed with SAMtools software, version 1.9 using mpileup algorithm [Li H, Handsaker B. et al. R (2009)]. The final BAM and pileup files obtained for each sample were used as input for inventor's in-house-developed software named MSID (MSI Detection tool). MSID comprises 3 algorithms: MSID Indels Counter, MSID Baseline and MSID Detector. The generated pileup files were processed using MSID Indels Counter to count the number of indels (insertions and deletions). MSID Indels Counter generates a VCF file for each sample containing all indels. As a first step, a baseline was generated using 20 MSS/pMMR control samples. This step was performed using MSID Baseline which involves MSID Indels Counter for indels counting at each locus of MSS/pMMR control samples. Subsequently, the mean and standard deviation (SD) of indels counts were calculated for each locus. Finally, a text file was generated containing the following information for each locus: name, genomic coordinates, mean and SD of indels count, instability threshold (mean+1SD) and number of MSS/pMMR control samples used in the calculation. Upon completion of the baseline, this was compared with samples to be tested. This step was performed by using MSID Indels Counter to get the indels number and MSID Detector for comparing loci in each sample with baseline. For each locus, if the indels count was above the baseline value, the corresponding locus was considered as “unstable”, otherwise the corresponding locus was considered as “stable”. Subsequently, MSID Detector gathered the number of unstable and stable loci and generated a MSID score (=unstable loci/total loci). The MSID score is ranging between 0 and 1. When using the 5-marker conventional panel, the sample status is considered “stable” if the MSID score≤0.2, “unstable” if the MSID score≥0.6, and “uncertain” if the MSID score=0.40 (and must be reassessed in this latter case). For analyses performed with the octaplex CaBio-MSID, the sample status is considered “stable” if the MSID score≤0.125, “unstable” if the MSID score≥0.5, and “uncertain” for MSID scores between 0.125 and 0.5, in the present example equal to (=) 0.25 and 0.375 (and must be reassessed in this latter case).

### Results

### Study Population and Analyses Performed

In this study, inventors investigated a total of 303 colorectal cancers (CRCs) and 88 endometrial cancers (ECs) selected from Poitiers University Hospital (FIG. 1).

Of the 303 CRC samples, 241 were pMMR/MSS and 62 dMMR/MSI tumors, and for EC samples, 60 were pMMR/MSS and 28 dMMR/MSI tumors (FIG. 1). CRC samples were obtained after biopsy (n=144) or colectomy (n=159) and EC samples were obtained after biopsy (n=7) or hysterectomy (n=81), with median tumor cell content of 65% for CRCs and 60% for ECs (18 missing data). Median age of patients at time of analysis was 74 and 72 years respectively for CRCs and ECs. Details about IHC and molecular alteration are reported in Table 2. Analysis of the CRC cohort indicate that 52.1% of samples carry only one molecular alteration, mainly on the KRAS gene (30.4%), without POLE mutation. In contrast, only 19.8% of CRC patients have multiple molecular alterations, with 43.6% on KRAS, 15.5% on BRAF and 14.2% on PIK3CA. Analysis of the EC cohort revealed a lower percentage of patients carrying single molecular alterations (27.3%) while 27.3% of patients were found with multiple molecular alterations, wherein 21.6% with PIK3CA mutation, 14.8% with CTNNB1 mutation and 5.7% with POLE mutation.

TABLE 2
Tumors characteristics: IHC and
most common molecular alterations

| | CRCs | ECs |
|---|---|---|
| | (n = 303) | (n = 88) |
| MMR IHC status | | |
| Loss of MLH1 and PMS2 expression | 37 (12.2%) | 14 (15.9%) |
| Loss of MSH2 and MSH6 expression | 4 (1.3%) | 3 (3.4%) |
| Isolated loss of PMS2 expression | 4 (1.3%) | 1 (1.1%) |
| Isolated loss of MSH6 expression | 1 (0.3%) | 1 (1.1%) |
| pMMR | 241 (79.6%) | 60 (68.2%) |
| Missing | 16 (5.3%) | 9 (10.3%) |
| Molecular alteration | | |
| Single molecular alterations | 158 (52.1%) | 24 (27.3%) |
| KRAS | 92 (30.4%) | 8 (9.1%) |
| NRAS | 10 (3.3%) | 1 (1.1%) |
| POLE | 0 | 0 |
| Other | 201 (66.3%) | 79 (89.8%) |
| Wild type | 79 (26.1%) | 21 (23.8%) |
| Missing | 6 (2.0%) | 19 (21.6%) |
| Patients with multiples molecular | 60 (19.8%) | 24 (27.3%) |
| alterations | | |
| Details of multiples molecular alterations | | |
| KRAS | 132 (43.6%) | 13 (14.8%) |
| BRAF | 47 (15.5%) | 0 |
| NRAS | 14 (4.6%) | 2 (2.3%) |
| POLE | 3 (1%) | 5 (5.7%) |
| ALK | 11 (3.6%) | 0 |
| PI3K | 43 (14.2%) | 19 (21.6%) |
| FGFR 2 or 3 | 5 (1.7%) | 12 (13.6%) |
| MET | 5 (1.7%) | 1 (1.1%) |
| CTNNB1 | 7 (2.3%) | 13 (14.8%) |

CRCs: colorectal cancers;
ECs: endometrial cancers;
MMR: MisMatch Repair;
pMMR: proficient MisMatch Repair;
IHC: immunohistochemistry

### Evaluation of the Conventional 5-Marker Panel for MSI-NGS Classification in Colorectal Cancers

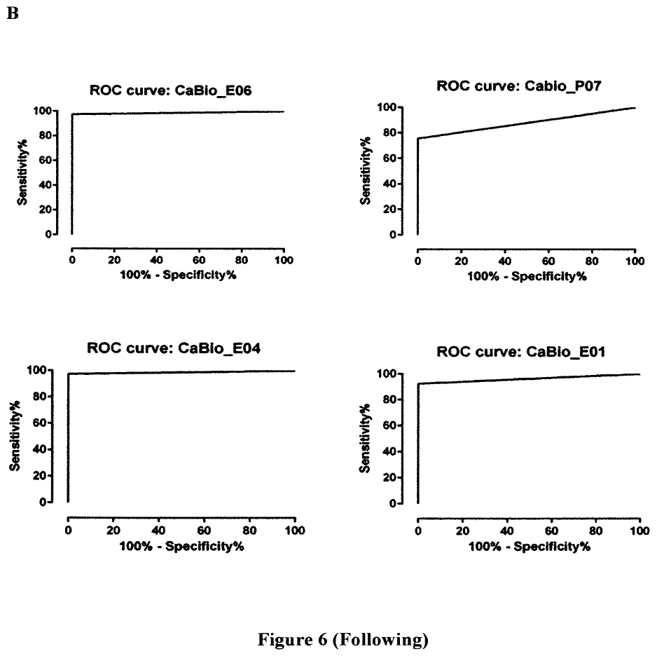

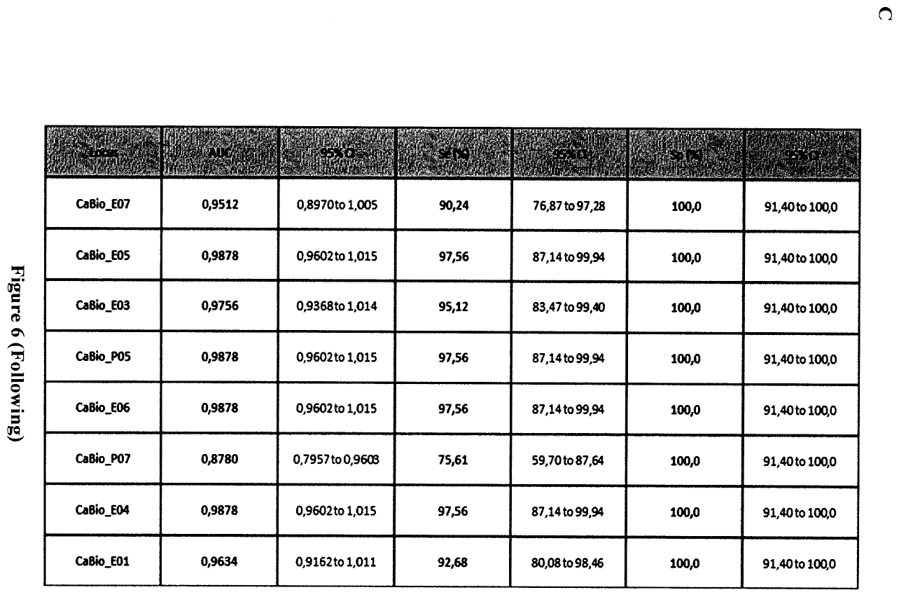

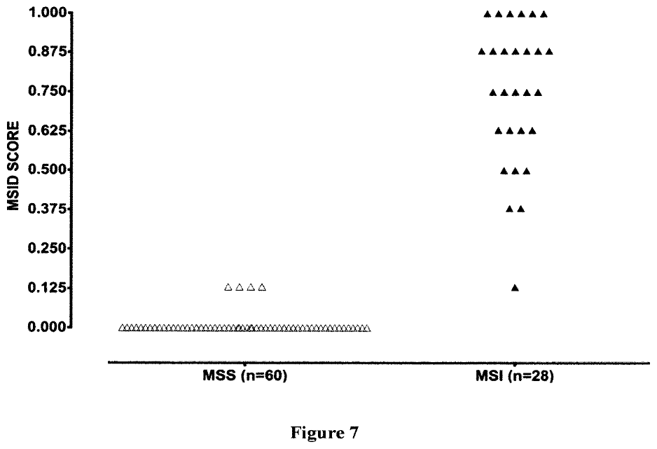

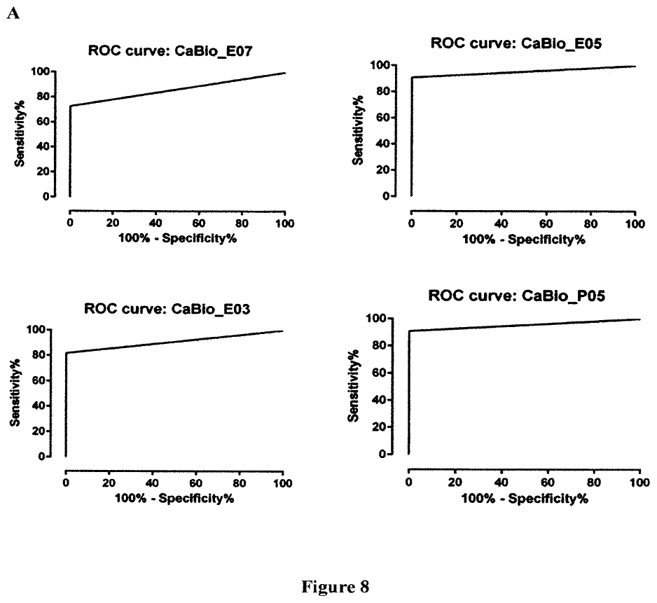

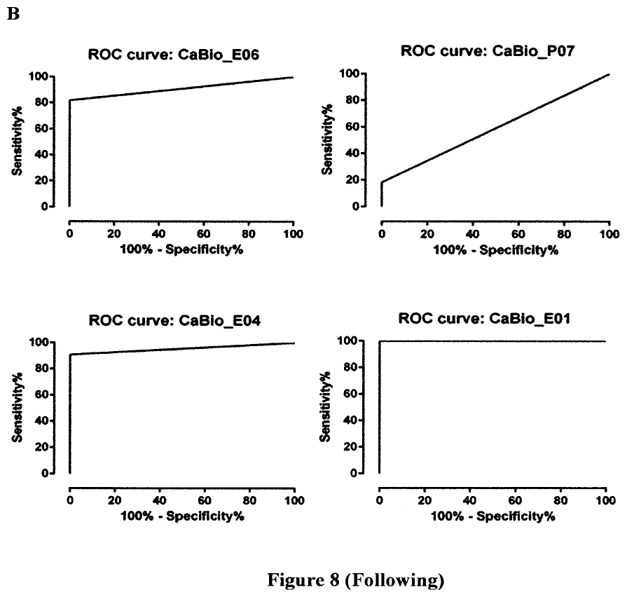

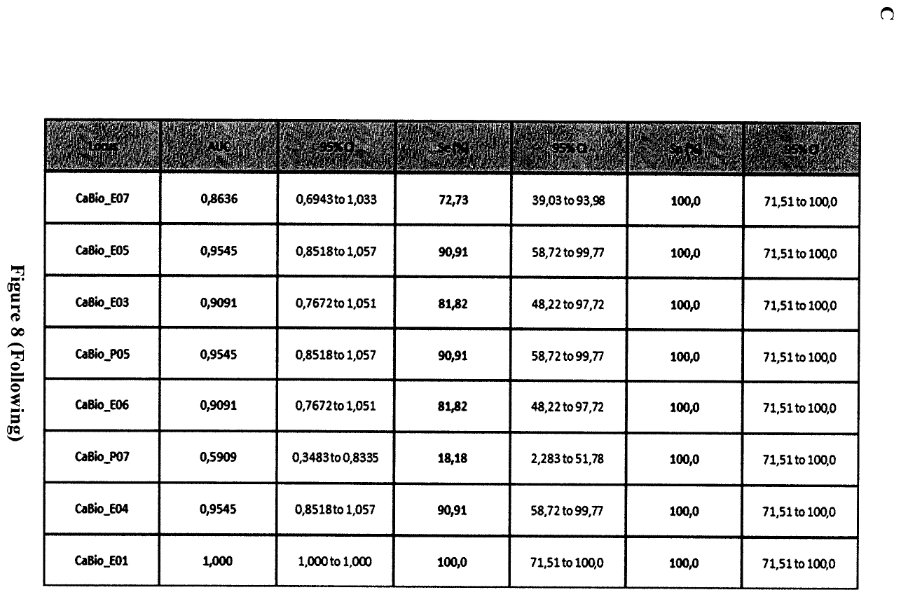

MSI-NGS classification of a CRCs cohort (303 tumors previously tested by reference methods IHC and PCR) with a conventional 5-marker panel using MSID confirmed the microsatellite status in 237/241 (98.3%) pMMR/MSS and 57/62 (91.9%) dMMR/MSI samples (FIG. 2). Among pMMR/MSS samples, 4 discordant cases (1.7%) were observed with MSID scores>0.2 (MSID scores=0.40, 2 unstable markers), whereas 5 discordant cases were observed for dMMR/MSI samples (8.1%), with MSID scores<0.6 (1 sample with MSID score=0, no unstable marker; 2 samples with MSID scores=0.2, 1 unstable marker; and 2 samples with MSID scores=0.4, 2 unstable markers).

This 5-marker MSI-NGS classification was not applied to EC samples, as preliminary tests showed low sensitivity for detecting dMMR/MSI status with 6 discordant cases (30%) out of 20 dMMR/MSI samples tested (data not shown), clearly highlighting the lack of sensitivity of this 5-marker conventional panel in detecting MSI in EC samples.

### Validation of the 8-Marker CaBio Panel for MSI-NGS Classification in Formalin-Fixed Paraffin-Embedded (FFPE) Tumor Samples

To overcome the poor performance of these 5 conventional microsatellite markers in non-colorectal cancers, particularly in ECs classification, inventors identified and characterized an 8-marker panel (CaBio octaplex) for the determination of microsatellite instability by MSI-NGS in colorectal as well as non-colorectal cancers (see Materials and Methods).

To validate the reproducibility of the 8-marker CaBio panel, 3 CRC samples were tested in triplicate, showing reproducible results for pMMR/MSS and dMMR/MSI tumors (FIG. 3).

Performance of the CaBio octaplex panel was evaluated in 56 tumors (30 CRCs and 26 ECs). FIG. 4 provides an overview of MSI-NGS results obtained for each microsatellite marker in CRCs (FIG. 4A) and ECs (FIG. 4B). Scoring results obtained by MSI-NGS with dMMR/MSI CRC and EC samples consistently show at least 4 unstable markers out of 8, suggesting an acceptable level of reliability for CaBio panel and MSID algorithm in the systemic determination of MSI status in both CRCs and ECs.

### Validation and Performance Evaluation of the 8-Marker CaBio Panel for MSI-NGS Classification in Colorectal Cancers

The octaplex CaBio-MSID was implemented in place of the conventional 5-marker panel to reassess the previous CRC cohort (303 tumors). MSID analysis confirmed the microsatellite status in 237/241 (98.3%) pMMR/MSS and 61/62 (98.4%) dMMR/MSI samples (FIG. 5). Discordances between MSI-NGS and reference methods were observed in 4 pMMR/MSS CRC samples (=1.7%) with MSID scores>0.125 (M6627 and M7047 samples with MSID scores=0.25, 2 unstable markers; M6623 and M6759 samples with MSID scores=0.375, 3 unstable markers), while only 1 discordant case (=1.6%) was observed for dMMR/MSI samples with MSID score<0.5 (M7093 sample with MSID score=0.25, 2 unstable markers). Consequently, all 5 discordant samples were classified as “uncertain” microsatellite status. IHC slides for M6627, M7047, M6623 and M6759 were re-read by different pathologist confirming the normal staining for MSH2, MSH6, MLH1 and PMS2 proteins. Re-reading of M7093 slide confirmed the isolated loss of PMS2, confirming the unstable microsatellite status of the tumor.

All 5 discordant samples were then reextracted using a different method ensuring higher purity level (Maxwell® 16 FFPE Plus LEV DNA Purification kit, Promega, see Materials and Methods). Reassessment of M6627, M7047, M6623 and M6759 after reextraction with Maxwell® kit revealed MSID scores between 0 and 0.125 (Table 3), confirming a stable microsatellite status, and confirming also the MSI status of M7093 (MSID score=0.5).

TABLE 3
Details of discordant CRC cases

| | | | | | | Maxwell ® |
|---|---|---|---|---|---|---|
| | | Tumor | | | Kapa extraction | extraction |
| | | cells | | MSI- | kit MSID | kit MSID |
| CRCs | Sample | (%) | IHC | PCR | score/conclusion | score/conclusion |
| M6623 | Sample 1 | 30% | *pMMR | MSS | 0.38 / uncertain | 0.125 / MSS |
| M6759 | Sample 1 | 5% | *pMMR | MSS | 0.38 / uncertain | 0 / MSS |
| M6627 | Sample 1 | 40% | *pMMR | MSS | 0.25 / uncertain | Failed |
| | Sample 2 | | | Not done | Not done | 0 / MSS |
| M7047 | Sample 1 | 20% | *pMMR | MSS | 0.25 / uncertain | 0.125 / MSS |
| M7093 | Sample 1 | 20% | Isolated | MSS | 0.25 / uncertain | 0.5 / MSI |
| | | | loss of | | | |
| | | | PMS2 | | | |

IHC: immunohistochemistry;
MSI: Microsatellite Instability;
PCR: Polymerase Chain Reaction
NGS: Next Generation Sequencing;
pMMR: proficient MisMatch Repair;
MSS: Microsatellite Stability;
MSID: MSI Detection tool
*no loss of expression for the 4 MMR proteins (MSH2, MSH6, MLH1 and PMS2)

The overall performance of the CaBio panel and MSID algorithm on CRCs were analyzed (Table 4 and FIG. 6).

TABLE 4
Performance characteristics of CaBio panel
for MSS/MSI diagnosis in CRC tumors

| Maximum unstable markers | | |
|---|---|---|
| for MSS classification (n/8) | Sensitivity | Specificity |
| 1 (MSID ≤ 0.125) | 100.0% | 98.3% |
| 2 (MSID ≤ 0.25) | 98.4% | 99.2% |
| 3 (MSID ≤ 0.375) | 98.4% | 100.0% |

MSS: microsatellite stability;
MSID: MSI Detection tool

When defining the upper limit for MSS classification various threshold have been considered to calculate the performance, for informational purposes values from 3 different thresholds (1, 2, or 3 unstable markers allowed for MSS classification) have been reported in Table 4. In the current study, MSID analysis has been performed by fixing the maximum threshold value to 1 unstable marker for MSS classification (MSID score≤0.125), showing a sensitivity=100% and a specificity=98.3%. Moreover, considering the uncertainty of some of the pMMR/MSS samples displaying 2 or 3 unstable markers (4 samples in this study), inventors decided to set the minimum threshold value to 4 unstable markers for MSI classification (MSID score≥0.5). Hence the overall performance obtained for CRCs indicates a sensitivity of 98.4%, a specificity of 98.4%, a positive predictive value of 93.8% and a negative predictive value of 99.6% when comparing CaBio-MSID results to pMMR/dMMR and MSS/MSI statuses determined from validated techniques.

### Validation and Performance Evaluation of the 8-Marker CaBio Panel for MSI-NGS Classification in Endometrial Cancers

To determine whether the octaplex CaBio-MSID could be suitable for the determination of microsatellite status in EC tumors, inventors performed MSI-NGS testing on a retrospective cohort of 88 EC samples. MSID analysis confirmed the microsatellite status in 60/60 (100%) pMMR/MSS and 25/28 (89.3%) dMMR/MSI samples (FIG. 7).

Table 5 and FIG. 8 show the overall performance of the CaBio panel and MSID algorithm on EC tumors. In an identical manner as MSS/MSI classification for CRC tumors, 3 different thresholds have been reported in Table 6. As for CRC tumors, the same maximum threshold for MSS classification and minimum threshold for MSI classification have been utilized. The overall performance obtained for ECs indicate a sensitivity of 89.3%, a specificity of 100%, a positive predictive value of 100% and a negative predictive value of 95.2% when comparing CaBio-MSID results to pMMR/dMMR and MSS/MSI statuses determined from validated techniques.

TABLE 5
Performance characteristics of CaBio panel
for MSS/MSI diagnosis in EC tumors

| Maximum unstable markers | | |
|---|---|---|
| for MSS classification (n/8) | Sensitivity | Specificity |
| 1 (MSID ≤ 0.125) | 96.4% | 100.0% |
| 2 (MSID ≤ 0.25) | 96.4% | 100.0% |
| 3 (MSID ≤ 0.375) | 89.3% | 100.0% |

MSS: microsatellite stability;
MSID: MSI Detection tool

### Discussion

The conventional 5-marker panel used for MSI status determination is long established and mostly used in combination with the MSI-PCR method. This 5-marker panel has been validated for CRCs only. An IVD (In Vitro Diagnosis) version of the 5-marker panel (OncoMate™ MSI Dx Analysis System), commercially manufactured by the Promega company is broadly used and shows satisfying performances (sensitivity of 97.3% and specificity of 97.2%) [PROMEGA MSI Analysis System, Version 1.2. Instructions for Use of Product]. However, this 5-marker panel has not been validated for non-CRC tumors. According to current projections there will be over 515,000 new cases of ECs in the world by the year 2030, 23.7% higher than 2020, and of these approximately 20 to 30% will need assessment of the MSI status (https://gco.iarc.fr). Since 2013, The Cancer Genome Atlas (TCGA) classification determined 4 histo-prognostic groups focusing on MSI status [Cancer Genome Atlas Research Network, Kandoth C et al.; Stelloo E etal.]. The “ultra-mutated” group (7%) concerns patients with a higher tumor mutational burden (TMB) and POLE mutations associated with a good prognosis. A “hyper-mutated” group (28%) consists of patients with high TMB and MSI status caused by MLH1 promoter methylation. A third group is called “low-copy number” (39%) and concerns patients with low TMB with mainly CTNNB1 mutation. The last group comprises patients with “serous-like” tumor (25%) with high somatic copy number alterations with mostly TP53 mutations [Stelloo E et al.]. Moreover, determination of the MSI status is a major new issue in EC due to the effectiveness of immunotherapy for this type of tumor [Berton D et al.; Marabelle A. et al.; Oaknin A. et al.].

In the context of the present invention, inventors used their octaplex CaBio-MSID to classify 303 CRCs and 88 ECs in comparison with reference tests (IHC and MSI-PCR pentaplex). The evaluation of the diagnostic performance of the octaplex CaBio-MSID for MSI status classification was satisfying in CRC tumors with a sensitivity of 98.4% and a specificity of 98.4%, as well as in EC tumors with a sensitivity of 89.3% and a specificity of 100%.

MSI-NGS classification with the octaplex CaBio-MSID on a CRC cohort showed a higher sensitivity (98.4% vs 97.3%) and specificity (98.4% vs 97.2%) compared to the OncoMate™ MSI Dx Analysis System. In the literature there are few studies implementing MSI-NGS algorithms, but most require matched normal sample which is a limitation when testing many patients to ensure timely delivery of the results [Ratovomanana T. et al.; Kautto E. A. et al.; Zhao L. et al.]. Among published studies, sensitivity levels for detecting MSI in CRCs are ranging from 76.1% to 100% while specificity levels fluctuate between 72.5% and 100% [Bacher J. W etal. (2005); Bacher J. W etal. (2004); Dietmaier W. et al.; Avvaru A. K et al.]. Inventors' results are advantageously ranking in the upper part of the range for both sensitivity and specificity.

Unlike ESMO guidelines for CRCs, there are currently no formally assessed microsatellite markers to be used for MSI testing in ECs. According to inventors' results, MSI-NGS classification with the octaplex CaBio-MSID shows better performance than commonly used methods such as the Idylla MSI test (automated PCR) with 89.3% vs 72.7% sensitivity (and 100% specificity for both) [Ukkola I. et al.]. Another study based on a probe capture method reported lower sensitivity than the octaplex CaBio-MSID with 75% sensitivity (and 100% specificity) [Waalkes A. et al.].

In practical terms the octaplex CaBio-MSID contains 8 markers and is therefore easily compatible with NGS, allowing assessment of microsatellite status concurrently to variant detection. The tests were reproduced with success by inventors using respectively only 7, 6, 5 and finally 4 markers arbitrarily chosen from the original set of 8 markers.

### REFERENCES

Amos W (September 2010). “Mutation biases and mutation rate variation around very short human microsatellites revealed by human-chimpanzee-orangutan genomic sequence alignments”. Journal of Molecular Evolution. 71 (3): 192-201).
André T, Shiu K. K, Kim T. W, Jensen B. V, Jensen L. H, Punt C, Smith D, . . . KEYNOTE-177 Investigators (2020). Pembrolizumab in Microsatellite-Instability-High Advanced Colorectal Cancer. New England J Med 383(23), 2207-2218.
Avvaru A. K, Sharma D, Verma A, Mishra R. K, Sowpati D. T (2020) MSDB: A Comprehensive, Annotated Database of Microsatellites. Nucleic Acids Res., 48, D155-D159.
Bacher J. W, Abdel Megid W. M, Kent-First M. G, Halberg R. B (2005) Use of Mononucleotide Repeat Markers for Detection of Microsatellite Instability in Mouse Tumors. Mol. Carcinog., 44, 285-292.
Bacher J. W, Flanagan L. A, Smalley R. L, Nassif N. A, Burgart L. J, Halberg R. B, Megid W. M. A, Thibodeau, S. N (2004) Development of a Fluorescent Multiplex Assay for Detection of MSI-High Tumors. Dis. Markers, 20, 237-250.
Berton D, Banerjee S. N, Curigliano G, Cresta S, Arkenau H.-T, Abdeddaim C, Kristeleit R. S, . . . André T (2021). Antitumor Activity of Dostarlimab in Patients with Mismatch Repair-Deficient/Microsatellite Instability-High Tumors: A Combined Analysis of Two Cohorts in the GARNET Study. J. Clin. Oncol. 39, 2564-2564.
Bonneville R, Krook M. A, Kautto E. A, Miya J, Wing M. R, Chen H.-Z, Reeser J. W, Yu L, Roychowdhury S (2017). Landscape of Microsatellite Instability Across 39 Cancer Types. JCO Precis. Oncol. PO.17.00073.
Buhard O, Suraweera N, Lectard A, Duval A, Hamelin R (2004) Quasimonomorphic Mononucleotide Repeats for High-Level Microsatellite Instability Analysis. Dis. Markers, 20, 251-257.
Cancer Genome Atlas Research Network, Kandoth C, Schultz N, Cheriack A. D, Akbani R, Liu Y, Shen H, . . . Levine DA (2013) Integrated Genomic Characterization of Endometrial Carcinoma. Nature 497, 67-73.
Church and Mitra; December 1999. “In situ localized amplification and contact replication of many individual DNA molecules.” Nucleic Acids Res. 27 (24).
Cunningham J. M, Christensen E. R, Tester D. J, Roche P. C, Burgart L. J, Thibodeau, S. N (1998)
Hypermethylation of the hMLH1 Promoter in Colon Cancer with Microsatellite Instability. Cancer Res 58 (15): 3455-3460.
Dietmaier W, Wallinger S, Bocker T, Kullmann F, Fishel R, Rüschoff, J (1997) Diagnostic Microsatellite Instability: Definition and Correlation with Mismatch Repair Protein Expression. Cancer Res., 57, 4749-4756.
Evrard C, Alexandre J (2021) Predictive and Prognostic Value of Microsatellite Instability in Gynecologic Cancer (Endometrial and Ovarian). Cancers 13, 2434.
Evrard C, Tachon G, Randrian V, Karayan-Tapon L, Tougeron D (2019) Microsatellite Instability: Diagnosis, Heterogeneity, Discordance, and Clinical Impact in Colorectal Cancer. Cancers 11(10), 1567.
Guyot D′Asnières De Salins A, Tachon G, Cohen R, Karayan-Tapon L, Junca A, Frouin E, Godet J, . . . Tougeron D (2021) Discordance between Immunochemistry of Mismatch Repair Proteins and Molecular Testing of Microsatellite Instability in Colorectal Cancer. ESMO Open, 6, 100120.
Forster P, Hohoff C, Dunkelmann B, Schirenkamp M, Pfeiffer H, Neuhuber F, Brinkmann B (March 2015). “Elevated germline mutation rate in teenage fathers”. Proceedings. Biological Sciences. 282 (1803): 20142898.
Griffiths, A. J. F., Miller, J. F., Suzuki, D. T., Lewontin, R. C. & Gelbart, W. M. (1996). Introduction to Genetic Analysis, 5th Edition. W. H. Freeman, New York.
Hempelmann J. A, Lockwood C. M, Konnick E. Q, Schweizer M. T, Antonarakis E. S, Lotan T. L,
Montgomery B, . . . Pritchard C. C (2018). Microsatellite Instability in Prostate Cancer by PCR or Next-Generation Sequencing. J. Immunother. Cancer, 6, 29.
Herbreteau G, Airaud F, Pierre-Noël E, Vallée A, Bézieau S, Théoleyre S, Blons H, . . . Denis M. G (2021). MEM: An Algorithm for the Reliable Detection of Microsatellite Instability (MSI) on a Small NGS Panel in Colorectal Cancer. Cancers, 13, 4203.
Jame P, Lagoda P J (October 1996). “Microsatellites, from molecules to populations and back”. Trends in Ecology & Evolution. 11 (10): 424-9.
Kautto E. A, Bonneville R, Miya J, Yu L, Krook M. A, Reeser J. W, Roychowdhury S (2016) Performance Evaluation for Rapid Detection of Pan-Cancer Microsatellite Instability with MANTIS. Oncotarget, 8, 7452-7463.
Klintschar M, Dauber E M, Ricci U, Cerri N, Immel U D, Kleiber M, Mayr W R (October 2004). “Haplotype studies support slippage as the mechanism of germline mutations in short tandem repeats”. Electrophoresis. 25 (20): 3344-8.
Kobayashi et al., Mol. Cell. Probes, 9:175-182, 1995.
Levine DA. Integrated genomic characterization of endometrial carcinoma. Nature [Internet]. 2013 [cited 2020 Oct. 6]; 497:67-73. Available from: World Wide Web: ncbi.nlm.nih.gov/pmc/articles/PMC3704730/.
Luchini C, Bibeau F, Ligtenberg M. J. L, Singh N, Nottegar A, Bosse T, Miller R, . . . Scarpa A (2019). ESMO Recommendations on Microsatellite Instability Testing for Immunotherapy in Cancer, and Its Relationship with PD-1/PD-L1 Expression and Tumour Mutational Burden: A Systematic Review-Based Approach. Ann. Oncol. 30, 1232-1243.
Li H, Durbin R (2009) Fast and Accurate Short Read Alignment with Burrows-Wheeler Transform. Bioinformatics, 25, 1754-1760.
Li H, Handsaker B, Wysoker A, Fennell T, Ruan J, Homer N, Marth G, . . . Durbin R (2009). The Sequence Alignment/Map Format and SAMtools. Bioinformatics, 25, 2078-2079.
Long D. R, Waalkes A, Panicker V. P, Hause R. J, Salipante S. J (2020). Identifying Optimal Loci for the Molecular Diagnosis of Microsatellite Instability. Clin. Chem. 66, 1310-1318.
Marabelle A, Le D. T, Ascierto P. A, Di Giacomo A. M, De Jesus-Acosta A, Delord J. P, Geva R, . . . Diaz L. A Jr (2020). Efficacy of Pembrolizumab in Patients With Noncolorectal High Microsatellite Instability/Mismatch Repair-Deficient Cancer: Results From the Phase II KEYNOTE-158 Study. J. Clin. Oncol. 38, 1-10.
Middha S, Zhang L, Nafa K, Jayakumaran G, Wong D, Kim H. R, Sadowska J, . . . Hechtman J. F (2017). Reliable Pan-Cancer Microsatellite Instability Assessment by Using Targeted Next-Generation Sequencing Data. JCO Precis. Oncol. 1, PO.17.00084.
Oaknin A, Gilbert L, Tinker A. V, Brown J, Mathews C, Press J, Sabatier R, . . . Pothuri B (2022) Safety and antitumor activity of dostarlimab in patients with advanced or recurrent DNA mismatch repair deficient/microsatellite instability-high (dMMR/MSI-H) or proficient/stable (MMRp/MSS) endometrial cancer: interim results from GARNET-a phase I, single-arm study. J Immunother Cancer. 10(1):e003777.
Ostrander E A, Jong P M, Rine J, Duyk G. Construction of small-insert genomic DNA libraries highly enriched for microsatellite repeat sequences. Proc Natl Acad Sci USA. 1992 Apr. 15; 89(8):3419-23. doi: 10.1073/pnas.89.8.3419. PMID: 1314388; PMCID: PMC48879.
PROMEGA MSI Analysis System, Version 1.2. Instructions for Use of Product.
Ratovomanana T, Cohen R, Svrcek M, Renaud F, Cervera P, Siret A, Letourneur Q, . . . Duval A (2021) Performance of Next-Generation Sequencing for the Detection of Microsatellite Instability in Colorectal Cancer With Deficient DNA Mismatch Repair. Gastroenterology, 161, 814-826.e7.
Saiki et al., Nature 324,163-166, 1986.
Stelloo E, Bosse T, Nout R. A, MacKay H. J, Church D. N, Nijman H. W, Leary A, . . . Creutzberg, C. L (2015). Refining Prognosis and Identifying Targetable Pathways for High-Risk Endometrial Cancer; a TransPORTEC Initiative. Mod Pathol, 28(6):836-44.
Stoneking et al., Am. J. Hum. Genet. 48:70-382, 1991.
Shia J (2008). Immunohistochemistry versus Microsatellite Instability Testing For Screening Colorectal Cancer Patients at Risk For Hereditary Nonpolyposis Colorectal Cancer Syndrome. J Mol. Diagn.10, 293-300.
Tougeron D, Mouillet G, Trouilloud I, Lecomte T, Coriat R, Aparicio T, Des Guetz G, Lécaille C, Artru P, Sickersen G, Cauchin E, Sefrioui D, Boussaha T, Ferru A, Matysiak-Budnik T, Silvain C, Karayan-Tapon L, Pagès J C, Vernerey D, Bonnetain F, Michel P, Taïeb J, Zaanan A. Efficacy of Adjuvant Chemotherapy in Colon Cancer With Microsatellite Instability: A Large Multicenter AGEO Study. J Natl Cancer Inst. 2016 Feb. 1; 108(7). doi: 10.1093/jnci/djv438. PMID: 26839356.
Trabucco S. E, Gowen K, Maund S. L, Sanford E, Fabrizio D. A, Hall M. J, Yakirevich E, . . . Sun J. X (2019). A Novel Next-Generation Sequencing Approach to Detecting Microsatellite Instability and Pan-Tumor Characterization of 1000 Microsatellite Instability-High Cases in 67,000 Patient Samples. J. Mol. Diagn. 21, 1053-1066.
Tytgat, Olivier; Gansemans, Yannick; Weymaere, Jana; Rubben, Kaat; Deforce, Dieter; Van Nieuwerburgh, Filip (2020). “Nanopore Sequencing of a Forensic STR Multiplex Reveals Loci Suitable for Single-Contributor STR Profiling”. Genes. 11 (4): 381.
Ukkola I, Nummela P, Pasanen A, Kero M, Lepistö A, Kytölä S, Bützow R, Ristimäki A (2021) Detection of Microsatellite Instability with Idylla MSI Assay in Colorectal and Endometrial Cancer. Virchows Arch., 479, 471-479.
Waalkes A, Smith N, Penewit K, Hempelmann J, Konnick E. Q, Hause R. J, Pritchard C. C, Salipante S. J (2018) Accurate Pan-Cancer Molecular Diagnosis of Microsatellite Instability by Single-Molecule Molecular Inversion Probe Capture and High-Throughput Sequencing. Clin. Chem., 64, 950-958.
Wang Y, Shi C, Eisenberg R, Vnencak-Jones C. L (2017) Differences in Microsatellite Instability Profiles between Endometrioid and Colorectal Cancers: A Potential Cause for False-Negative Results?JMD 19(1), 57-64.
Weber J L, Wong C (August 1993). “Mutation of human short tandem repeats”. Human Molecular Genetics. 2 (8): 1123-8.
Zhao L, Shan G, Li L, Yu Y, Cheng G, Zheng X (2020) A Robust Method for the Rapid Detection of Microsatellite Instability in Colorectal Cancer. Oncol. Lett., 20, 1982-1988.

1-14. (canceled)

15. A method of analyzing a set of at least four microsatellite loci of human DNA selected from the group comprising:
CABIO-P05, defined as a 21 T repeat located at 14q23.1 and starting at position chr14: 58359108,
CABIO-P07, defined as a 21 T repeat located at 7q32 and starting at position chr7: 131478596,
CABIO-E01, defined as a 22 T repeat located at Xq22.3 and starting at position chrX: 106849221,
CABIO-E03, defined as a 23 T repeat located at Xq21.2 and starting at position chrX: 85268269,
CABIO-E04, defined as a 25 T repeat located at 14q32.3 and starting at position chr14: 103574079,
CABIO-E05, defined as a 22 T repeat located at 2p11.2 and starting at position chr2: 86456417,
CABIO-E06, defined as a 23 T repeat located at 4q23 and starting at position chr4: 99216136, and
CABIO-E07, defined as a 21 T repeat located at 20p13 and starting at position chr20: 290564, with reference to the Homo sapiens reference genome assembly from Genome Reference Consortium human Build 38 patch release 14 (GRCh38.p14).

16. The method of claim 15, wherein the method comprises a step of sequencing the set of at least four microsatellite loci.

17. The method of claim 16, wherein the method comprises a step of co-amplifying the set of at least four microsatellite loci with primers, wherein at least one of the primers has a nucleic acid sequence selected from the group of primer sequences identified by SEQ ID NO: 9-24, or a sequence having at least 90% identity thereto.

18. The method of claim 17, wherein the method involves Next-Generation Sequencing (NGS) and further optionally comprises assessing the expression, or loss of expression, of at least one of the MLH1, MSH2, MSH6 and PMS2 proteins of the MisMatch Repair (MMR) system.

19. A method of assessing the microsatellite stability (MSS) or microsatellite instability (MSI) status of a tumor comprising the steps of:
a) counting the number of indel(s) in at least four microsatellite loci in a sample of tumor's DNA, wherein the microsatellite loci are selected from the group comprising CABIO-P05, CABIO-P07, CABIO-E01, CABIO-E03, CABIO-E04, CABIO-E05, CABIO-E06 and CABIO-E07;
b) comparing, for each locus of the at least four microsatellite loci, the number of indel(s) to a reference number of indel(s) for the locus, and determining the MSI status of said locus, a locus being considered as unstable if the indels count is equal to or above (≥) the reference value for the locus and as stable if the indels count is below (<) the reference value for the locus;
c) calculating a MSID score, ranging between 0 and 1, consisting of the total number of unstable loci on the total number of loci; and
d) determining that the MSI status of the tumor is unstable if the MSID score is equal to or above (≥) 0.5; stable if the MSID score is equal to or below (≤) 0.125, and uncertain if the MSID score is between 0.125 and 0.5; and, optionally
e) if the MSID score is between 0.125 and 0.5, repeating steps a)-d) until the MSI status can be determined, wherein:
CABIO-P05 is defined as a 21 T repeat located at 14q23.1 and starting at position chr14: 58359108,
CABIO-P07 is defined as a 21 T repeat located at 7q32 and starting at position chr7: 131478596,
CABIO-E01 is defined as a 22 T repeat located at Xq22.3 and starting at position chrX: 106849221,
CABIO-E03 is defined as a 23 T repeat located at Xq21.2 and starting at position chrX: 85268269,
CABIO-E04 is defined as a 25 T repeat located at 14q32.3 and starting at position chr14: 103574079,
CABIO-E05 is defined as a 22 T repeat located at 2p11.2 and starting at position chr2: 86456417,
CABIO-E06 is defined as a 23 T repeat located at 4q23 and starting at position chr4: 99216136, and
CABIO-E07 is defined as a 21 T repeat located at 20p13 and starting at position chr20: 290564, with reference to the Homo sapiens reference genome assembly from Genome Reference Consortium human Build 38 patch release 14 (GRCh38.p14).

20. The method of claim 19, wherein the method is for assessing precancerous and/or cancerous cells for microsatellite instability; for detecting a predisposition to develop a cancer; for evaluating the prognostic of a cancer; for monitoring cancer progression or regression; for predicting, evaluating or monitoring the response to a treatment of cancer; for selecting the appropriate treatment of cancer for a subject in need thereof; for selecting patients capable of responding to a treatment of cancer; or for selecting patients for enrollment in a clinical trial for the treatment of cancer.

21. The method of claim 19, wherein the method is a partially or fully computer-implemented method.

22. The method of claim 21, wherein the method uses a classifier trained to determine the microsatellite stable (MSS) or unstable (MSI) status of a tumor with a method comprising:
a) providing a training set of microsatellite loci, each locus being obtained from a DNA sequence of interest, or preprocessed information obtained from said training set, as input to the classifier, said training set comprising i) stable (MSS) microsatellites loci, or sub-sequences thereof, obtained from proficient MisMatch Repair system (pMMR) or MSS cells, known as having a microsatellite stable status or phenotype, and ii) unstable (MSI) microsatellites loci, or sub-sequences thereof, obtained from deficient MisMatch Repair system (dMMR) or MSI cells, known as having a microsatellite unstable status or phenotype;
b) generating an output of the classifier for each microsatellite locus, said output classifying the microsatellite locus input as having a stable (MSS) or an unstable (MSI) status or phenotype; and
c) evaluating the classifier's accuracy for distinguishing between a stable (MSS) and an unstable (MSI) status or phenotype by comparing, for each microsatellite locus, the output of the classifier to the known actual phenotype of the microsatellite locus or to a reference number of indels for the microsatellite locus;
wherein the classifier is considered as an accurate classifier to determine the MSS or MSI status or phenotype of a tumor, if it exhibits an accuracy in counting indels for each of the microsatellite loci with a resolution of 1 base pair.

23. A computer-implemented method of training a classifier for determining the microsatellite stability (MSS) or microsatellite instability (MSI) status or phenotype of a tumor, wherein the method comprises:
a) providing a training set of microsatellite loci, each locus being obtained from a DNA sequence of interest, or preprocessed information obtained from said training set, as input to the classifier, said training set comprising i) stable (MSS) microsatellites loci, or sub-sequences thereof, obtained from proficient MisMatch Repair system (pMMR) or MSS cells, known as having a microsatellite stable status or phenotype, and ii) unstable (MSI) microsatellites loci, or sub-sequences thereof, obtained from deficient MisMatch Repair system (dMMR) or MSI cells, known as having a microsatellite unstable status or phenotype;
b) generating an output of the classifier for each microsatellite locus, said output classifying the microsatellite locus input as having a stable (MSS) or an unstable (MSI) status or phenotype; and
c) evaluating the classifier's accuracy for distinguishing between a stable (MSS) and an unstable (MSI) status or phenotype by comparing, for each microsatellite locus, the output of the classifier to the known actual phenotype of the microsatellite locus or to a reference number of indels for the microsatellite locus;
wherein the classifier is considered as an accurate classifier to determine the MSS or MSI status or phenotype of a tumor, if it exhibits an accuracy in counting indels for each of the microsatellite loci with a resolution of 1 base pair.

24. The method of claim 23, wherein the unstable (MSI) microsatellites loci used to prepare the training set are obtained from the DNA of a cancerous tumor cell, the cancer being selected from a colorectal cancer, an endometrial cancer, a prostate cancer, a renal cancer, a gastric cancer, a cholangiocarcinoma, a pancreatic cancer, a lung cancer, or a brain cancer.

25. The method of claim 23, wherein the classifier is selected from random forest (RF) classifier, Support Vector Machine (SVM) classifier, decision tree classifier, K-nearest neighbor classifier (KNN), logistic regression classifier, nearest neighbor classifier, Gaussian mixture model (GMM) classifier, nearest centroid classifier, linear regression classifier, a neural network, an artificial neural network, deep neural network, convolutional neural network, or fully connected neural network.

26. A computing system comprising:
a memory storing at least one instruction of a classifier trained according to the method of claim 23, and
a processor accessing to the memory for reading said instruction(s) and executing the method comprising the steps of:
a) counting the number of indel(s) in at least four microsatellite loci in a sample of tumor's DNA, wherein the microsatellite loci are selected from the group comprising CABIO-P05, CABIO-P07, CABIO-E01, CABIO-E03, CABIO-E04, CABIO-E05, CABIO-E06 and CABIO-E07;
b) comparing, for each locus of the at least four microsatellite loci, the number of indel(s) to a reference number of indel(s) for the locus, and determining the MSI status of said locus, a locus being considered as unstable if the indels count is equal to or above (≥) the reference value for the locus and as stable if the indels count is below (<) the reference value for the locus;
c) calculating a MSID score, ranging between 0 and 1, consisting of the total number of unstable loci on the total number of loci; and
d) determining that the MSI status of the tumor is unstable if the MSID score is equal to or above (≥) 0.5; stable if the MSID score is equal to or below (≤) 0.125, and uncertain if the MSID score is between 0.125 and 0.5; and, optionally
e) if the MSID score is between 0.125 and 0.5, repeating steps a)-d) until the MSI status can be determined.

27. A kit for analyzing microsatellite loci of human genomic DNA comprising oligonucleotide primers for co-amplifying a set of microsatellite loci of human genomic DNA, characterized in that it comprises at least two pairs of primers suitable for the amplification of, and/or at least two oligonucleotide probes for the detection of, CABIO-P05, CABIO-P07,
CABIO-E01, CABIO-E03, CABIO-E04, CABIO-E05, CABIO-E06 and/or CABIO-E07 or any combination thereof, and optionally a thermostable polymerase and/or control DNA isolated from normal non-cancerous biological material and/or lacking mismatch repair genes, wherein:
CABIO-P05 is defined as a 21 T repeat located at 14q23.1 and starting at position chr14: 58359108,
CABIO-P07 is defined as a 21 T repeat located at 7q32 and starting at position chr7: 131478596,
CABIO-E01 is defined as a 22 T repeat located at Xq22.3 and starting at position chrX: 106849221,
CABIO-E03 is defined as a 23 T repeat located at Xq21.2 and starting at position chrX: 85268269,
CABIO-E04 is defined as a 25 T repeat located at 14q32.3 and starting at position chr14: 103574079,
CABIO-E05 is defined as a 22 T repeat located at 2p11.2 and starting at position chr2: 86456417,
CABIO-E06 is defined as a 23 T repeat located at 4q23 and starting at position chr4: 99216136, and
CABIO-E07 is defined as a 21 T repeat located at 20p13 and starting at position chr20: 290564, with reference to the Homo sapiens reference genome assembly from Genome Reference Consortium human Build 38 patch release 14 (GRCh38.p14).
